(19) United States
(12) Patent Application Publication
(10) Pub. No.: US 2004/0030034 A1
(43) Pub. Date: Feb. 12, 2004

(54) Triggered response compositions

(76) Inventors: Ching-Jen Chang, Ambler, PA (US); Richard Thomas Gray, Levittown, PA (US); Hailan Guo, Warrington, PA (US); Barry Weinstein, Dresher, PA (US)

Correspondence Address: ROHM AND HAAS COMPANY, PATENT DEPARTMENT, 100 INDEPENDENCE MALL WEST, PHILADELPHIA, PA, 19106-2399, US

(21) Appl. No.: 10/629,137

(22) Filed: Jul. 29, 2003

Related U.S. Application Data
(60) Provisional application No. 60/399,904, filed on Jul. 31, 2002.

(51) Int. Cl. A61K 9/00; C 08L 1 1/12

(52) U.S. Cl. 524/543; 424/400; 524/881

(57) ABSTRACT

This invention provides a triggered response composition in the form of a barrier material and a delivery device that includes one or more polyelectrolytes in contact with an aqueous system that is stable and insoluble in a liquid medium and that exhibits one or more chemical/physical responses in the liquid medium, wherein the chemical/physical response of the composition is triggered upon a change of ionic strength in the liquid medium.

The present invention relates to compositions that are capable of producing a chemical or physical response that is triggered upon exposing the compositions to fluidized and liquid media containing one or more or a series of triggering events, each triggering event encompassing a chemical/physical process or property of the medium. In particular, it relates to regulating the stability of polyelectrolyte compositions in aqueous and non-aqueous systems by one or more triggering events in such systems that result in the dissolution, disintegration, deformation, swelling and/or dispersion of the polyelectrolyte compositions at a specified time, wherein triggering events are brought about by marked alterations in ionic strength and other chemical and/or physical changes in the system in addition to ionic strength. The present invention is further directed to devices containing triggered responsive compositions useful for the delivery of active ingredients and beneficial agents in a fluid medium to an environment of use.

It is often desirable to provide compositions and devices that deliver or provide controlled release of one or more active ingredients/beneficial agents to an environment of use.

International Publication Patent No. WO 00/17311 discloses a coated a detergent active encapsulated with a coating material which enabling a delayed release of the detergent active in to a washing solution, the coating material being insoluble in a washing solution having a pH equal to or greater than 10 at 25° C., yet being soluble in a washing solution having a pH equal to or less than 9 at 25° C. The coating materials disclosed include amines, waxes, Schiff base compounds and mixtures thereof U.S. Patent Application Publication No. 2001/0031714 A1 discloses a laundry detergent portion having two or more detersive components of which at least two are released into the wash liquor at different times, the portion including at least one temperature or pH switch to provide controlled release of the detersive components. The switch materials disclosed include waxes, amino alkyl methacrylate copolymers and polymers containing pyridine groups.

Encapsulated active ingredients having a pH sensitive coating material to delay release of the actives, however, suffer a number of limitations. The use of pH sensitive materials to achieve triggered release of detergent actives to rinse cycle is difficult because of the problem of the active or beneficial agent prematurely leaking into the liquid environment of use. As a consequence, all or most of the actives either disperse prematurely or are subsequently removed before their intended use in the environment of interest, preventing the controlled release of the desired actives in single or multiple-environment processes or the desired actives are released in amounts that are not effective in achieving the beneficial effect of the active as a result of controlled release. In addition, it is difficult to precisely control the release of active ingredients in a complex system such as, for example, a fabric laundry system that encompasses a broad spectrum of soil containing loads, numerous ingredients, varying water purity, varying amounts of water hardness, varying wash conditions, varying detergent concentration, a broad spectrum of washing machine designs, cycle lengths, washing and rinsing temperatures practiced by users worldwide. Major disadvantages in controlling the delivery of active ingredients and/or beneficial agents associated with current controlled release materials include incompatibility of ingredients, inability to release certain active components at or within defined time periods, premature release of active ingredients, and inability to control the stability of or trigger a change in the stability of the materials employed.

The use of materials sensitive only to changes in pH to achieve a site specific delivery of an active ingredient is difficult because typically 10 to 30% of the active ingredient is released prematurely due to degradation of the materials at high pH. It is therefore desirable to provide compositions whose stability can be altered by chemically and/or physically triggered events and whose response is to effect the controlled release of a wide variety of active ingredients and beneficial agents. Inventors have discovered compositions including one or more polyelectrolytes whose stability can be altered by changes in ionic strength and compositions including one or more trigger means in addition to ionic strength would be of significant utility as triggered response barrier materials, encapsulating agents and devices for the triggered delivery of fabric care active ingredients, personal care active ingredients, pharmaceutically beneficial agents and other related beneficial agents.

One practical solution to the problem of controlled release of one or more active ingredients/beneficial agents in an aqueous or a non-aqueous system was to use triggered response polyelectrolyte compositions whose polymer properties such as stability and solubility were a function of changes in one or more chemical and/or physical properties of the aqueous or non-aqueous system in which the polyelectrolyte was dispersed. Adjusting one or more chemical and/or physical properties of an aqueous system, such as the ionic strength, trigger the polyelectrolyte to respond by destabilizing, dissolving, disintegrating, deforming, swelling and/or dispersing in to the aqueous system. The ionic strength triggering event includes one or more changes in the ionic strength of the aqueous system. One class of triggered response compositions responds by destabilizing, dissolving, disintegrating, swelling and/or dispersing in to the aqueous system under relatively low ionic strength conditions while remaining stable and insoluble under relatively high ionic strength conditions. Alternatively, a separate class of triggered response compositions responds by remaining stable and insoluble in an altered or separate aqueous system under relatively low ionic strength conditions while destabilizing, dissolving, disintegrating, deforming, swelling or dispersing into the aqueous system under relatively high ionic strength conditions. Active ingredients and beneficial agents contained therein or encapsulated by triggered response barriers and devices constructed from such polyelectrolyte compositions are retained in order to protect such actives and agents in an aqueous system including but not limited to a fabric laundry wash cycle, an aqueous system-substrate interface such as skin, using a personal care delivery device and/or a pharmaceutical delivery device, and which then can be triggered or manipulated to produce a desired release of actives via dissolution, degradation, disintegration, swelling and/or dispersion of the polyelectrolytes during a subsequent process, such as fabric laundry rinse cycle, rinsing skin, or perspiration on skin, the chemical/physical polymer response triggered through alterations of one or more or a series of changes in the chemical and/or physical properties of the aqueous system in addition to ionic strength including: water hardness, acid strength and concentration, base strength and concentration, surfactant concentration, pH, buffer strength and buffer capacity, temperature, hydrogen bonding, solvents, hydrogen bonding solvents, organic solvents, osmotic pressure, polymer swelling, charge density, degree of neutralization of acidic and basic functional groups, degree of quaternization of basic functional groups, dilution, viscosity, electrochemical potential, conductivity, ion mobility, charge mobility, diffusion, surface area, mechanical forces, pressure, shearing forces, radiation and combinations thereof.

### SUMMARY

The present inventors have discovered classes of polyelectrolytes that are usefully employed in the present invention. The polyelectrolytes include carefully selected monomer compositions and specifically designed polymeric structures such that the chemical and/or physical response of the polymers is triggered by changes in one or more properties of both the polyelectrolyte and the fluidized or liquid medium in which they are in contact with (e.g. dispersed in) as a consequence of one or more parameters including: types and amounts of acidic or basic monomers, degree of neutralization of the acidic or basic monomers, types and amounts of amphoteric monomers, types and amounts of non-ionic vinyl surfactants, types and amounts of radiation responsive functional groups, types and amounts of residual unsaturated functional groups, types and amounts of chemically reactive functional groups, types and amounts of electrically responsive functional groups, types and amounts of electrochemically active functional groups, types and amounts of radiation responsive (ultraviolet, visible, infrared, X-rays) functional groups, ionic strength of the system, ion concentration in the system, the pH of the system, temperature of the system and surfactant concentration of the system.

Suitable polyelectrolytes include for example alkali soluble/swellable emulsion (ASE) polymers, hydrophobically modified alkali soluble/swellable emulsion (HASE) polymers, acid soluble/swellable emulsion polymers, hydrophobically modified acid soluble/swellable emulsion polymers, acidic homopolymers, copolymers and salts thereof; basic homopolymers, copolymers and salts thereof; poly(quaternized amine) homopolymers, copolymers and salts thereof; amphoteric polymers; anionic, cationic and amphoteric polysaccharide homopolymers, copolymers and salts thereof; anionic, cationic and amphoteric polysaccharides derivatives; anionic, cationic and amphoteric polypeptide homopolymers, copolymers and salts thereof; anionic, cationic and amphoteric polypeptide derivatives; chemically modified polypeptide homopolymers, copolymers and salts thereof; nucleic acid homopolymers, copolymers and salts thereof; chemically modified nucleic acids, naturally derived nucleic acids, enzymes, synthetic and naturally derived proteins, gelatins, lignosulfonic acid homopolymers, copolymers and salts thereof; ionene homopolymers, copolymers and salts thereof; anionic, cationic and amphoteric polyester homopolymers, copolymers and salts thereof; anionic, cationic and amphoteric polyurethane homopolymers, copolymers and salts thereof; copolymer combinations of recited homopolymers, copolymers and salts thereof; ionic and non-ionic micelles; stoichiometric and non-stoichiometric interpolymer combinations of the recited homopolymers, copolymers and salts thereof; polymer matrices of the recited homopolymers, copolymers and salts thereof; physical blends of the recited homopolymers, copolymers and salts thereof; recited homopolymers, copolymers and salts thereof having cationic, anionic and amphoteric components grafted thereon, and combinations thereof.

Inventors have further discovered that such polyelectrolytes form effective barrier materials for dispersing, sequestering, adhering to, depositing on, surrounding, encapsulating and/or forming a matrix with one or more active ingredients in an aqueous system and that the stability of the barrier materials can be usefully manipulated to respond to changes in one or more chemical and/or physical properties of the aqueous system in addition to ionic strength including, for example, ion concentration, surfactant concentration, acid strength and concentration, base strength and concentration, pH, buffer strength and capacity, temperature, hydrogen bonding, solvents, hydrogen bonding solvents, organic solvents, osmotic pressure, polymer swelling, charge density, degree of neutralization, dilution, viscosity, electrochemical potential, conductivity, ion mobility, charge mobility, diffusion, surface area, mechanical forces, radiation and combinations thereof.

In one embodiment, the polyelectrolyte compositions of the present invention, in an aqueous system under relatively high ionic strength conditions, are sufficiently stable and form effective barriers to contain, encapsulate and/or form a matrix with one or more active ingredients/beneficial agents. Exposing the compositions to an aqueous system under relatively low ionic strength conditions, triggers instability in the compositions such that the active ingredients are rapidly dispersed in the aqueous system. The triggered response compositions of the present invention obviate the limitations noted above and provide new compositions, devices, and processes for delivering controlled release of one or more active ingredients/beneficial agents to an environment of use.

Accordingly, there is provided a triggered response composition comprising: one or more polyelectrolytes in contact with a fluidized or liquid medium that is stable in the liquid medium that exhibits one or more chemical/physical responses wherein the chemical/physical response of the composition is triggered upon one or more ionic strength changes to the liquid medium. The polyelectrolyte comprises: (a) one or more acidic, basic or amphoteric monomers; (b) one or more non-ionic vinyl monomers; optionally, (c) one or more non-ionic vinyl surfactant monomers; and optionally (d) one or more polyethylenically unsaturated monomers or cross-linking agents, wherein the chemical/physical response of the composition in addition to ionic strength changes is dependent on one or more parameters selected from the group consisting of (i) the type and amounts of acidic monomers, (ii) the type and amounts of basic monomers, (iii) the degree of neutralization of the acidic and basic monomers, including the degree of quaternization of the basic monomers, (iv) the type and amounts of non-ionic monomers, (v) the type and amounts of non-ionic vinyl surfactant monomers, (vi) the type and amounts of polyethylenically unsaturated monomers, (vii) the type and amounts of cross-linking agents, (viii) and combinations thereof.

In one preferred embodiment, the polyelectrolyte is one or more alkali soluble/swellable emulsion polymers comprising: (a) 15-70 weight percent of one or more acidic monomers; (b) 15-80 weight percent of one or more non-ionic vinyl monomers; (c) 0-30 weight percent of one or more non-ionic vinyl surfactant monomers; and optionally (d) 0.001-5 weight percent of one or more polyethylenically unsaturated monomers. Moreover, the polyelectrolyte compositions are stable and insoluble in an aqueous system at relatively high ionic strength and the composition disperses, dissolves, deforms, swells or degrades in an aqueous system at relatively low ionic strength or when the ionic strength of the aqueous system in contact with the composition is lowered. The aqueous system optionally contains hydrogen bonding solvents and/or organic solvents and the chemical/physical response of the composition is triggered by one or more parameters in addition to ionic strength selected from: ion concentration, surfactant concentration, acid strength and concentration, base strength and concentration, pH, buffer strength and capacity, temperature, hydrogen bonding, solvent, hydrogen bonding solvents, organic solvents, osmotic pressure, polymer swelling, charge density, degree of neutralization, dilution, viscosity, electrochemical potential, conductivity, ion mobility, charge mobility, polymer chain entanglement and the combinations thereof. Preferably, the HASE polymer comprises: (a) 20-50 weight percent of one or more acidic monomers; (b) 20-70 weight percent of one or more non-ionic vinyl monomers; (c) 2-20 weight percent of one or more non-ionic vinyl surfactant monomers; and optionally, (d) 0.05 to 0.5 weight percent of one or more polyethylenically unsaturated monomers.

In a separate embodiment, the polyelectrolyte includes one or more alkali soluble/swellable emulsion polymers comprising: (a) 15-70 weight percent of one or more acidic monomers; (b) 15-80 weight percent of one or more non-ionic vinyl monomers; and optionally (c) 0.001-5 weight percent of one or more metal cross-linking agents.

In another embodiment, the polyelectrolyte is one or more acid soluble/swellable emulsion polymers comprising: (a) one or more basic monomers; (b) one or more non-ionic vinyl monomers; (c) one or more non-ionic vinyl surfactant monomers; and optionally, (d) one or more polyethylenically unsaturated monomers or cross-linking agents; wherein the basic monomers may be quaternized before or after polymerization.

In yet another embodiment, the polyelectrolyte is one or more amphoteric emulsion polymers comprising: (a) one or more acidic and basic monomers; (b) one or more non-ionic vinyl monomers; (c) one or more non-ionic vinyl surfactant monomers; and optionally, (d) one or more polyethylenically unsaturated monomers, metal and/or other cross-linking agents.

In a separate embodiment, the polyelectrolyte is one or more Morez® polymers comprising: (a) 15-70 weight percent of one or more acidic monomers; (b) 15-80 weight percent of one or more non-ionic vinyl monomers; and optionally (c) 0.001-5 weight percent of one or more polyethylenically unsaturated monomers, metal and/or other cross-linking agents.

In a separate embodiment, the polyelectrolyte is one or more polymers comprising: (a) 15-70 weight percent of one or more acidic monomers; (b) 15-80 weight percent of one or more non-ionic vinyl monomers; (c) 0.5-30 weight percent of one or more polyethylenically unsaturated or functionalized vinyl monomers and optionally (d) 0.001-5 weight percent of one or more polyethylenically unsaturated monomers, metal and/or other cross-linking agents.

In a separate embodiment, the polyelectrolyte is one or more polymers comprising: (a) 15-70 weight percent of one or more basic monomers; (b) 15-80 weight percent of one or more non-ionic vinyl monomers; (c) 0.5-30 weight percent of one or more polyethylenically unsaturated or functionalized vinyl monomers and optionally (d) 0.001-5 weight percent of one or more polyethylenically unsaturated monomers, metal and/or other cross-linking agents; wherein the basic monomers may be quaternized before or after polymerization.

Secondly, there is provided a triggered response barrier composition comprising: one or more polyelectrolytes in contact with a liquid medium, wherein the barrier composition surrounds, encapsulates or forms a matrix with one or more active ingredients and is stable in the liquid medium; wherein the barrier exhibits one or more chemical/physical responses selected from dispersing, disintegrating, degrading, dissolving, destabilizing, deforming, swelling, softening, melting, conducting electrical current, spreading, absorbing, adsorbing, flowing and combinations thereof; wherein the chemical/physical response of the composition is triggered upon one or more chemical/physical changes to the liquid medium; and wherein the barrier composition is capable of releasing the active ingredients to the liquid medium as a result of the triggered response. One or more triggering events in the form of chemical/physical changes to the system in contact with or containing the polymer or the polymer itself are usefully employed in the present invention.

In one preferred embodiment, the chemical/physical changes to the liquid medium are one or more changes in ionic strength. In another embodiment, the chemical/physical changes to the liquid medium are changes in ion concentration. In another embodiment, the chemical/physical changes to the liquid medium are changes in ionic strength and pH. In another embodiment, the chemical/physical changes to the liquid medium are changes in ionic strength and temperature. In another embodiment, the chemical/physical changes to the liquid medium are changes in ionic strength, pH and temperature. In another embodiment, the chemical/physical changes to the liquid medium are changes in ionic strength and mechanical shearing forces (e.g. agitation, convection). In yet another separate embodiment, the chemical/physical changes to the polymer dispersed in or in contact with the liquid medium are changes in the amount and/or intensity of ultraviolet/visible radiation. In accordance with the invention, the chemical/physical changes to the polymer dispersed in or in contact with the liquid medium are a plurality of triggered chemical/physical changes in the liquid medium.

### DETAILED DESCRIPTION

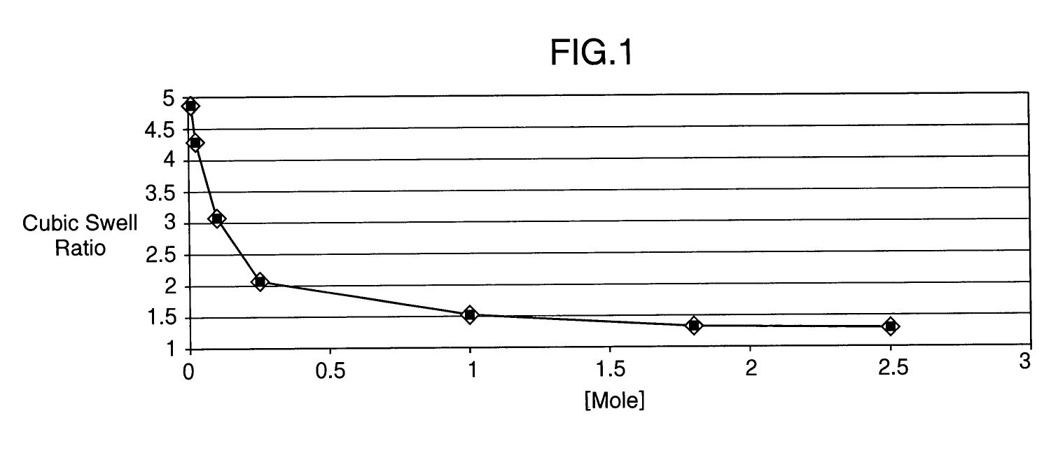

FIG. 1. Depicts Cubic Swell Ration of PEL Free-standing Films in Aqueous NaCl Solution at pH 12.

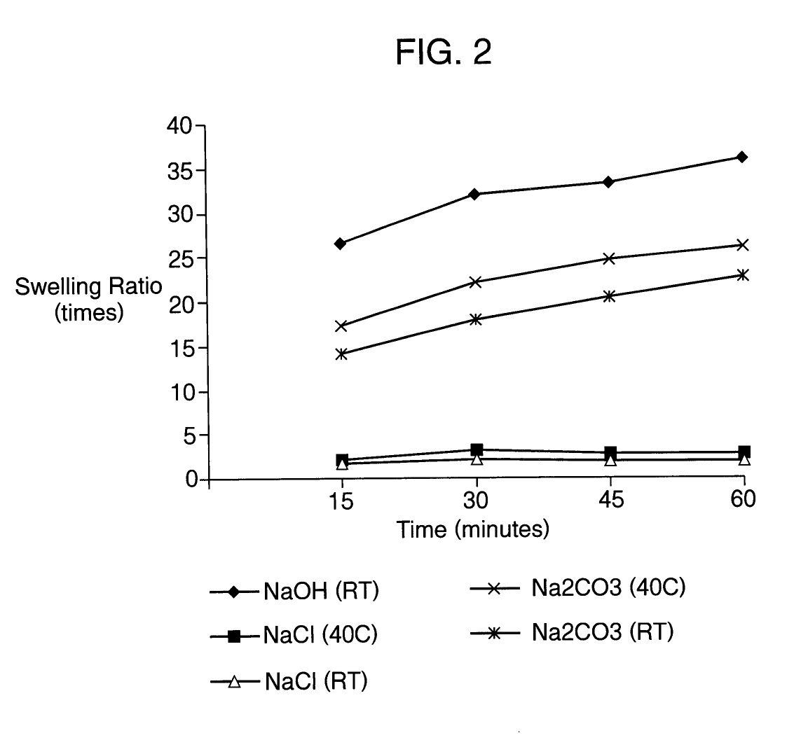

FIG. 2. Depicts Swell Rates of PEL (Composition D) Films in 0.1 M Salt and Base Solutions.

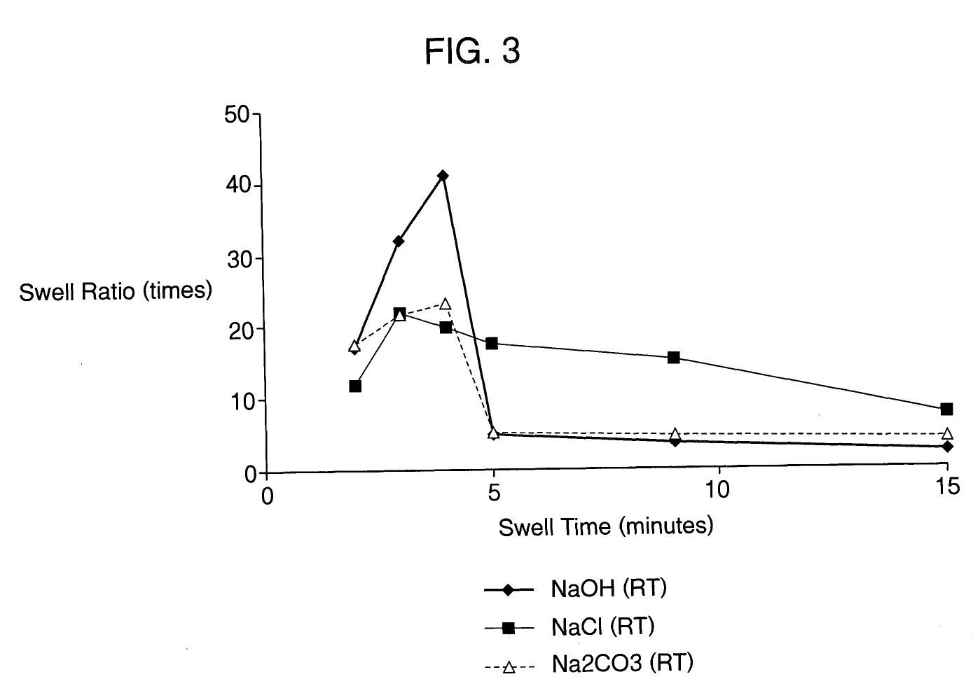

FIG. 3. Depicts Swell Rates of PEL (Composition D) Films in 0.001 M Salt and Base Solutions.

There is provided a device for the triggered release of one or more active ingredients to an environment of use comprising:

(a) one or more active ingredients;

(b) one or more additives; and

(c) a barrier composition comprising one or more ionic strength responsive polyelectrolytes;

wherein the barrier composition surrounds, encapsulates or forms a matrix with one or more active ingredients; wherein the barrier composition is stable in a liquid medium; wherein the barrier exhibits one or more chemical/physical responses in the liquid medium; wherein the chemical/physical response of the composition is triggered upon one or more ionic strength changes to the liquid medium; and wherein the device is capable of releasing the active ingredients to the environment of use as a result of the triggered response of the barrier composition.

There is also provided a process for triggering the release of one or more active ingredients to an environment of use comprising the steps of:

(a) surrounding, encapsulating or forming a matrix with one or more active ingredients with an ionic strength responsive barrier composition, the barrier being substantially impermeable to releasing the active ingredients when in contact with a liquid medium and remaining insoluble in the liquid medium when not triggered to respond; and

(b) altering chemical/physical properties of the liquid medium;

wherein the barrier composition disperses, destabilizes, degrades, disintegrates, dissolves, deforms or swells and becomes substantially permeable, thereby triggering the release of the active ingredients into the environment of use.

The term “polyelectrolyte” as it relates to the present invention refers to a polymer or macromolecular compound, in contact with a liquid medium, containing a plurality of ionized and/or ionizable groups within the polymer as a result of the polymerization of one or more monomers having ionized and/or ionizable groups. The polyelectrolyte is preferably in contact with an aqueous system or with a non-aqueous system including solvents are capable of solvating the plurality of ions that comprise the polyelectrolyte. Suitable aqueous systems include for example water, water incorporating hydrogen bonding solvents, polar solvents and organic solvents. Typical polar compounds include for example both organic and inorganic acids, bases and buffers. Typical organic solvents include but are not limited to alcohols, polyalkylene glycols, poly(alcohols), ethers, poly(ethers), amines, poly(amines), carboxylic acids, oligomeric carboxylic acids, organophosphorus compounds, and combinations thereof. A fluidized or liquid medium refers to any aqueous system, non-aqueous system or system of free flowing solids. Suitable liquid mediums include for example aqueous dispersions, aqueous solutions, aqueous dispersions containing one or more solvents and free-flowing dispersions of polymer solids. Non-aqueous systems are also usefully employed in the invention, including for example those containing solvents that can solvate ions and charged groups of polyelectrolytes.

Polyelectrolytes usefully employed in the invention include for example exclusively cationic groups, exclusively anionic groups or may be amphoteric, containing a combination of cationic and anionic groups. The individual ionized and/or ionizable components of the polyelectrolyte include for example weak or strong acidic groups, such as carboxylic, sulphonic, phosphonic and phosphinic groups respectively; strong or weak basic groups such as primary amines, secondary amines, tertiary amines, and phosphines respectively; and amphoteric groups such as amino acids and alternating acidic and basic groups of a copolymer. Suitable examples of polyelectrolytes usefully employed in the invention include for example alkali soluble/swellable emulsion (ASE) polymers, hydrophobically modified alkali soluble/swellable emulsion (HASE) polymers, acid soluble/swellable emulsion polymers, hydrophobically modified acid soluble/swellable emulsion polymers, acidic homopolymers, copolymers and salts thereof, such as polycarboxylic acids, Morez® polymers, polycarboxylates, poly(acrylic acid), poly(methacrylic acid) and polyacrylates; basic homopolymers, copolymers and salts thereof, such as polyamines, poly(amideamino) acrylates, and poly(amino)acrylamides; poly(quaternized amine) homopolymers, copolymers and salts thereof, such as quaternized poly(amino) acrylates, amphoteric emulsion polymers such as poly(amino acids) and poly (amino acid) acrylate emulsion polymers; anionic, cationic and amphoteric polysaccharide homopolymers, copolymers and salts thereof; anionic, cationic and amphoteric polysaccharides derivatives; anionic, cationic and amphoteric polypeptide homopolymers, copolymers and salts thereof; anionic, cationic and amphoteric polypeptide derivatives; chemically modified polypeptide homopolymers, copolymers and salts thereof; nucleic acid homopolymers, copolymers and salts thereof; chemically modified nucleic acids, naturally derived nucleic acids, enzymes, synthetic and naturally derived proteins, gelatins, lignosulfonic acid homopolymers, copolymers and salts thereof; ionene homopolymers, copolymers and salts thereof; anionic, cationic and amphoteric polyester homopolymers, copolymers and salts thereof; anionic, cationic and amphoteric polyurethane homopolymers, copolymers and salts thereof; copolymer combinations of recited homopolymers, copolymers and salts thereof; physical blends of the recited homopolymers, copolymers and salts thereof; recited homopolymers, copolymers and salts thereof having cationic, anionic and amphoteric components grafted thereon, and combinations thereof Suitable polyelectrolytes (PEL) of the present invention include both synthetic, natural and chemically modified polyelectrolytes. Preferred polyelectrolyte include alkali and acid soluble/swellable emulsion polymers, amphoteric emulsion polymers, poly(amino acid) polymers and Morez® polymers.

Synthesis of synthetic PEL including acid and alkali soluble emulsion polymers are carried out by well known and conventional methods of polymer chemistry including for example free-radical polymerization in homogeneous and heterogeneous phases, ionic polymerization, polycondensation, polyaddition and polymer modification. The isolation of preformed PEL from natural sources and/or products are carried out by conventional separation techniques including for example the chemical modification of isolated non-ionic polymer biopolymers and combinations of both methods. The chemical structures and useful properties of PEL within the scope of the present invention are further varied and altered by the synthesis of copolymers containing different amounts of ionic and non-ionic monomer units and non-ionic vinyl surfactant monomer units. This includes hydrophobic as well as hydrophilic co-monomers, which function to impart very different properties in aqueous systems and very different intermolecular and intramolecular interactions in the aqueous systems, and very different interactions on solid surfaces and at interfaces with the aqueous systems and combinations thereof.

Synthetic PEL are prepared by methods including for example chain growth processes such a free radical polymerization using ethylenically unsaturated monomers containing unstrained and strained ring systems via ionic processes, step growth processes and by modification of preformed polymers. Included with free radical polymerization for example are PEL homopolymers, copolymers, random copolymers, alternating copolymers, block copolymers, graft copolymers, blends of one or more homopolymers, blends of copolymers, and combinations thereof. PEL chemical structure and PEL macromolecular architecture can be controlled or modified by the various types and properties of the monomer units, including polymerization conditions such as initiators and other variables. Step-growth condensation polymerization are useful for the synthesis of natural PEL such as polypeptides and polynucleotides.

The PEL usefully employed in the present invention are characterized by one or more of the following properties/parameters including for example (i) types and amounts of acidic monomers, (ii) types and amounts of basic monomers, (iii) the degree of neutralization of the acidic and basic monomers, including the degree of quaternization of the basic monomers, (iv) the type and amounts of non-ionic monomers, (v) the type and amounts of non-ionic vinyl surfactant monomers, (vi) the type and amounts of polyethylenically unsaturated monomers, (vii) the type and amounts of cross-linking agents, (viii) PEL macromolecular architectures such as linear and branched structures, (ix) PEL electrochemical properties such as ion mobility and ionic conductivity, (x) PEL macromolecular polydispersity and related properties such as Mn and Mw, (xi) and combinations thereof.

The term “triggered response” as it relates to the present invention refers to regulating, manipulating or altering one or more chemical/physical properties of a polymer composition in contact with a liquid medium by triggering changes in or through alteration the chemical/physical properties of the liquid medium.

Typical chemical/physical properties of the liquid medium in addition to ionic strength include for example surfactant concentration, acid strength and concentration, base strength and concentration, pH, buffer strength and capacity, temperature, hydrogen bonding, hydrogen bonding solvents, organic solvents, osmotic pressure, dilution, viscosity, electrochemical potential, conductivity, ion mobility, charge mobility, polymer chain entanglement, diffusion, surface area, emulsion particle size, mechanical forces, radiation and combinations of such parameters. The inventors have discovered that the solubility, swellability and stability response of liquid soluble/swellable triggered response polymer compositions, barrier materials and devices in the liquid medium can be triggered by altering or changing the ionic strength and/or one or more additional parameters of the liquid medium, the liquid medium preferably an aqueous or non aqueous system.

Alkali soluble/swellable emulsion (ASE) polymers are polyelectrolytes based on acid-containing emulsion polymers disclosed in U.S. Pat. Nos. 3,035,004 and 4,384,096 (HASE polymers) and Great Britain Pat. No. 870,994. The inventors have discovered that adjusting the type and level of acid monomers and co-monomers in ASE and HASE polymers coupled with the degree of neutralization to achieve optimum charge density to afford polymers that are stable, having a low degree of swelling and insoluble in an aqueous system of relatively high ionic strength. The polymers can be characterized as incorporating an ionic strength trigger or referred to as ionic strength sensitive polymers. Changes in the ionic strength of the aqueous system to lower levels results in the a polymer that rapidly disperses, dissolves or swells to a significant extent in the aqueous system.

Accordingly, in a preferred embodiment, there is provided a triggered response composition comprising: one or more polyelectrolytes in contact with an aqueous system that is stable and that exhibits one or more chemical/physical responses selected from dispersing, degrading, dissolving, destabilizing, disintegrating, deforming, swelling, softening, melting, spreading, and flowing; wherein the chemical/physical response of the composition is triggered upon one or more ionic strength changes to the aqueous system. The polyelectrolyte is one or more alkali soluble emulsion polymers comprising: (a) 15-70 weight percent of one or more acidic, basic or amphoteric monomers; (b) 15-80 weight percent of one or more non-ionic vinyl monomers; (c) 0-30 weight percent of one or more non-ionic vinyl surfactant monomers; and optionally (d) 0-5 weight percent of one or more polyethylenically unsaturated monomers.

The ASE and HASE polymers of the present invention are typically prepared using standard emulsion polymerization techniques under acidic conditions such that the carboxylic acid groups are in protonated form to insolubilize the polymer and afford a liquid emulsion. PEL of this class are also referred to as anionic PEL. When added as a liquid colloidal dispersion, the finely divided ASE polymer particles dissolve almost instantly upon pH adjustment. The degree of neutralization, the type and amounts of both acidic monomers and non-ionic surfactant groups of the HASE polymers can be controlled precisely, affording ionic strength sensitive polymers whose stability, swell properties and solubility depend on the ionic strength of the aqueous system. The polymer compositions usefully employed in the present invention include one or more trigger means, namely for example an ionic strength triggering condition. The ease of handling, metering, and dispersing ASE and HASE polymers, the rapid solubilization and optimization of charge density on neutralized acidic functional groups by controlled pH adjustment, and the highly desirable film forming and barrier properties make ASE and HASE polymers a most effective and efficient barrier composition for a wide variety of applications including regulated release devices for personal care actives, household actives, and pharmaceutically beneficial agents, encapsulating compositions, matrices and devices that effect the controlled release of beneficial agents and active ingredients, sensor materials and sensing devices, imaging and diagnostic agents, materials and devices for separations, molecular recognition, tracing and biological molecular conjugate assays.

The HASE polymers of this invention include three components, as disclosed in U.S. Pat. No. 4,384,096: (a) 15-70 weight percent of one or more acidic monomers, (b) 15-80 weight percent of one or more non-ionic vinyl monomers, (c) 0-30 weight percent of one or more non-ionic vinyl surfactant monomers, and optionally (d) 0.01-5 weight percent of one or more polyethylenically unsaturated monomers. It has been discovered that the effectiveness of ASE and HASE polymers as ionic strength and pH responsive compositions for triggered release is critically dependent on the following components: (i) the type and amounts of acidic monomers, (ii) the degree of neutralization of the acidic monomers, and (iii) the type and amounts of non-ionic vinyl surfactant monomers, (iv) the type and amounts of non-ionic vinyl surfactant monomers, (v) the type and amounts of polyethylenically unsaturated monomers, (vi) the pH of the aqueous system and (vii) combinations thereof.

The acid monomers provide the requisite ionic strength and pH responsiveness and the degree of neutralization of the acidic monomers is critical in optimizing the charge density of the acidic groups. The non-ionic vinyl monomers provide an extended polymer backbone structure and added hydrophobic balance. The non-ionic vinyl surfactant monomers provide a bound surfactant. All four components contribute to preparing ionic strength sensitive polymers and barrier compositions whose stability, swell properties and solubility depend on the ionic strength of the aqueous system. Within the stated limits, the proportions of the individual monomers can be varied to achieve optimum properties for specific triggered release applications.

The ASE and HASE polymers require 15-70 weight percent based on total monomer content of one or more acidic monomers selected from the group consisting of C₃-C₈ α,β-ethylenically unsaturated carboxylic acid monomers such as acrylic acid, methacrylic acid, maleic acid, crotonic acid, itaconic acid, fumaric acid, aconitic acid, vinyl sulfonic acids and vinyl phosphonic acids, acryloxypropionic acid, methacryloxypropionic acid, monomethyl maleate, monomethyl fumarate, monomethyl itaconate and the like and combinations thereof Acrylic acid (AA) or methacrylic acid (MAA) or a mixture thereof are preferred. Mixtures of AA or MAA with itaconic or fumaric acid are suitable and mixtures of crotonic and aconitic acid and half esters of these and other polycarboxylic acids such as maleic acid with C₁-C₄ alkanols are also suitable, particularly if used in minor amount in combination with acrylic or methacrylic acid. For most purposes, it is preferable to have at least about 15 weight percent and most preferably from about 20-50 weight percent of acidic monomers. However, polycarboxylic acid monomers and half esters can be substituted for a portion of the acrylic or methacrylic acid, e.g., about 1-15 weight percent based on total monomer content.

To provide a stable aqueous dispersion and a desirable hydrophobic:hydrophilic balance needed for the ASE and HASE polymers of the present invention requires about 15-80 weight percent of one or more co-polymerizable non-ionic monomers selected from the group consisting of C₂-C₁₈ α,β-ethylenically unsaturated monomers, C₁-C₈ alkyl and C₂-C₈ hydroxy alkyl esters of acrylic and methacrylic acid including ethyl acrylate, ethyl methacrylate, methyl methacrylate, 2-ethylhexyl acrylate, butyl acrylate, butyl methacrylate, 2-hydroxyethyl acrylate, 2-hydroxybutyl methacrylate; styrene, vinyltoluene, t-butylstyrene, isopropylstyrene, and p-chlorostyrene; vinyl acetate, vinyl butyrate, vinyl caprolate; acrylonitrile, methacrylonitrile, butadiene, isoprene, vinyl chloride, vinylidene chloride, and the like. In practice, a mono vinyl ester such as methyl acrylate, ethyl acrylate, butyl acrylate is preferred.

These monomers, of course, must be co-polymerizable with the acidic monomers and vinyl surfactant monomers. Normally about 15-80 weight percent, and preferably about 20-70 weight percent of nonionic vinyl monomer, based on total weight of monomers, is used in preparing ASE polymers.

The third monomer component is about 0.1-30 weight percent based on total monomer content of one or more non-ionic vinyl surfactant monomers, preferably selected from the group consisting of an acrylic or methacrylic acid ester of a C₁₂—C₂₄ alkyl monoether of a polyalkylene glycol having at least 2 oxyalkylene units therein, preferably having at least 6 to 70 oxyalkylene units. More preferred are the acrylate and methacrylate surfactant esters selected from the group consisting of: alkyl phenoxy poly(ethyleneoxy)ethyl acrylates and methacrylates; alkoxy poly(ethyleneoxy)ethyl acrylates and methacrylates; wherein the ethyleneoxy unit is about 6-70. Preferable monomers may be defined by the general formula H₂C═C(R)—C(O)—O(CH₂CH₂O)ₙR′ wherein R is H or CH₃, the latter being preferred, n is at least 2, and preferably has an average value of at least 6, up to 40 to 60 and even up to 70 to 100 and R′ is a hydrophobic group, for example, an alkyl group or an alkyl phenyl group having 12 to 24 carbon atoms or having an average of 12 to 24 or more carbon atoms.

Typical vinyl surfactant monomers are the acrylic or methacrylic acid esters of certain nonionic surfactant alcohols. Such surfactant esters are known in the art. For example, Junas et al. U.S. Pat. No. 3,652,497 describe the use of alkylphenoxypoly(ethyleneoxy)ethyl acrylates in preparing several other polymeric surfactant thickeners. Dickstein U.S. Pat. No. 4,075,411 describes several processes for preparing such vinyl surfactant esters including the acid catalyzed condensation of commercially available nonionic polyoxyalkylene surfactant alcohols such as alkylphenoxypoly(ethyleneoxy)ethyl alcohol and block-polymeric glycols with acrylic, methacrylic, crotonic, maleic, fumaric, itaconic or aconitic acid. Alternate esterification methods including alcoholysis and transesterification are also described. Other suitable vinyl surfactant esters can be prepared from monoethers of mixed or heteropolymeric ethyleneoxypropyleneoxy-butyleneoxy polyglycols such as described in Patton U.S. Pat. No. 2,786,080. Additional surfactant alcohols which can be esterified for use herein are given in “McCutcheon's Detergents and Emulsifiers” 1973, North American Edition, Allured Publishing Corp., Ridgewood, N.J. 07450.

Certain of these vinyl surfactant monomer esters, i.e., those defined by the Formula are useful in preparing the HASE polymers described herein. It is essential that the surfactant be incorporated in the liquid emulsion product by copolymerization. Advantageously the requisite surfactant esters are prepared by the direct acid catalyzed esterification of the appropriate surfactant alcohol with an excess of the carboxylic acid monomer used as Component A. The resulting mixture with excess acid can be used directly in the copolymerization provided that at least 30 percent, and preferably 50-70 percent or more, of the surfactant alcohol in the mixture is esterified. The vinyl surfactant ester can also be recovered, purified by conventional means using an appropriate inhibitor such as hydroquinone or p-tert-butylcatechol to prevent undesired homopolymerization, and then used to prepare HASE polymers.

It has been found that the balance of acidic monomers to non-ionic monomers is an important factor in the triggered release response and performance of the resulting ASE and HASE polymers used in barrier or encapsulating compositions.

Optionally, the ASE and HASE polymers include a small amount of at least one polyethylenically unsaturated monomer, to provide a polymer having a network structure. One or more polyethylenically unsaturated monomers may be combined with the monomers during the polymerization process or may be added after the polymerization of monomers. Suitable examples include allyl methacrylate (ALMA), ethylene glycol dimethacrylate (EGDMA), butylene glycol dimethacrylate (BGDMA), diallyl phthalate (DAP), methylenebisacrylamide, pentaerythritol di-, tri- and tetra-acrylates, divinyl benzene, polyethylene glycol diacrylates, bisphenol A diacrylates and combinations thereof Low levels of the polyethylenically unsaturated monomers are preferred, since levels greater than about 5% by weight tend to over cross-link the polymer or provide a polymer network structure such that their effectiveness in the invention markedly decreases. Preferred amounts of the polyethylenically unsaturated monomers range from 0.01 to 5% by weight based on the total weight of the polymer, more preferably from 0.05 to 0.5% by weight based on the total weight of the polymer.

Optionally, the ASE and HASE polymers also include a small amount of at least one metal and/or alkaline earth cross-linking agent, to provide a polymer having a more rigid structure and better mechanical properties. One or more metal and/or alkaline earth cross-linking agents may be combined with the monomers during the polymerization process or may be added after the polymerization of monomers. Suitable metal and/or alkaline earth cross-linking agents include for example alkaline earth ions of calcium, magnesium and barium, transition metal ions of iron, copper and zinc. Other suitable examples such as aluminum ions are described in U.S. Pat. No. 5,319,018. Preferred amounts of the metal and/or alkaline earth cross-linking agents range from 0.01 to 5 % by weight based on the total weight of the polymer, more preferably from 0.05 to 0.5% by weight based on the total weight of the polymer.

Alkali soluble/swellable emulsion (ASE) polymers are polyelectrolytes based on acid-containing emulsion polymers disclosed in U.S. Pat. Nos. 3,035,004 and Great Britain Pat. No. 870,994. Alkali soluble resins (ASR) are polyelectrolytes based on acid-containing polymers and conventional methods used to prepare them are described in U.S. Pat. No. 5,830,957. ASR include polymers referred to as Morez® polymers. The inventors have discovered that adjusting the type and level of acid monomers and co-monomers in ASE and ASR polymers coupled with the degree of neutralization to achieve optimum charge density to afford polymers that are stable, having a low degree of swelling and insoluble in an aqueous system of relatively high ionic strength. The polymers can be characterized as incorporating an ionic strength trigger or referred to as ionic strength, base strength or dilution responsive polymers. Changes in the ionic strength, base strength or dilution of the aqueous system to lower levels results in the a polymer that rapidly disperses, dissolves or swells to a significant extent in the aqueous system.

The alkali swellable/soluble polymers of the present invention are typically prepared using standard emulsion polymerization techniques under acidic conditions such that the carboxylic acid groups are in protonated form to insolubilize the polymer and afford a liquid emulsion. When added as a liquid colloidal dispersion, the finely divided polymer particles dissolve almost instantly upon pH adjustment. Alkali swellable/soluble resins are typically prepared by a heated and pressurized reactor (also referred to as a continuous tube reactor or Morez® reactor) and conventional methods used to prepare them are described in U.S. Pat. No. 5,830,957. ASR include polymers referred to as Morez® polymers. The degree of neutralization, the type and amounts of both acidic monomers and non-ionic surfactant groups of the polymers of both ASE polymers and ASR can be controlled precisely, affording ionic strength, base strength or dilution sensitive/responsive polymers whose stability, swell properties and solubility depend on the ionic strength, base strength or dilution of the aqueous system. The polymer compositions are also referred to as incorporating ionic strength, base strength and dilution triggering conditions. The ease of handling, metering, and dispersing the polymers, the rapid solubilization and optimization of charge density on neutralized acidic functional groups by controlled pH adjustment, and the highly desirable film forming and barrier properties make alkali soluble/swellable emulsion polymers and alkali soluble/swellable resins a most effective and efficient barrier composition for a wide variety of applications including regulated release devices for floor care and household actives. Both ASE polymers and ASR are usefully employed in the present invention for preparing, processing, and/or fabricating encapsulating compositions that include at least one active ingredient/beneficial agent; whereby the chemical/physical triggers included within the encapsulated composition and activated on contact with chemical/physical changes in an environment of use (e.g an aqueous system) effect the controlled release of beneficial agents and active ingredients to the environment of use.

The ASE polymers and ASR of this invention include the following monomer components: (a) 5-70 weight percent of one or more acidic monomers and (b) 30-95 weight percent of one or more non-ionic vinyl monomers. Optionally, the ASE polymers may include a third component (c) 0.01-5 weight percent of one or more metal cross-linking agents or one or more polyethylenically unsaturated monomers. It has been discovered that the effectiveness of the polymers as ionic strength, base strength or dilution responsive compositions for triggered release is critically dependent on the following components: (i) the type and amounts of acidic monomers, (ii) the degree of neutralization of the acidic monomers, and (iii) the type and amounts of non-ionic vinyl monomers, (iv) the type and amounts of polyethylenically unsaturated monomers or the type and amounts of metal and other cross-linking agents, (v) the pH of the aqueous system and (vi) combinations thereof.

Alkali swellable/soluble resins are typically prepared by a heated and pressurized reactor (also referred to as a continuous tube reactor or Morez® reactor) and conventional methods used to prepare them are described in U.S. Pat. No. 5,830,957. Final ASR physical characteristics are dependant upon monomer content, initiator type and quantity, reaction time and reaction temperature. ASR include polymers referred to as Morez® polymers. ASR have weight average molecular weights that range from 1,000 to 20,000. Polymer acid number can also be varied by depending upon the desired degree of water solubility or dispersibility. Resin acid numbers range from between 50 to 300. Aqueous solutions or dispersions of ASR may be prepared by simply mixing the resins with a solution of water and at least one base. The monomer feed to these reactors contains from 5 to 15% by weight solvent to control in-process viscosity. Typical solvents include but are not limited to alkylene glycols including dipropylene glycol monomethyl ether (DPM) and diethylene glycol monomethyl ether (DE). Some solvent becomes esterified in the ASR product and most of the residual solvent (@ 50% by weight) is removed by stripping. The level of incorporated solvent effects the performance of the dispersant as an aqueous emulsion or when employed as a stabilizer in an emulsion polymerization. The ASR are typically supplied as ammonia neutralized aqueous solutions, though they are also prepared as sodium hydroxide neutralized solutions as well. The resulting ASR dispersions can be formulated into dispersions or emulsions containing no volatile organic compounds (VOC). Both hydrophilic and hydrophobic ASR can be prepared. Hydrophobic monomers used to prepare hydrophobic or oil soluble ASR are described in U.S. Pat. Nos. 5,521,266 and 5,830,957. Hydrophobic monomers used to prepare hydrophobic or oil soluble ASR are described in U.S. Pat. No. 4,880,842.

Multistage ASR are also usefully employed in the present invention wherein a partially or fully neutralized ASR emulsion is used as a first stage (core stage) and a partially cross-linked to fully cross-linked ASR and/or an ASR having a substantially different Tg (typically but not exclusively higher than the core stage) is used as a second stage (shell stage). “Multiphase” polymer or resin refers to polymer particles with at least one inner phase or “core” phase and at least one outer phase or “shell” phase. The phases of the polymers are incompatible. Incompatible refers to the fact that the inner and the outer phases are distinguishable using analytical characterization techniques known to those having skill in the art. Typically, such techniques include but are not limited to electron microscopy and staining that differentiate or distinguish the phases. The morphological configuration of the phases of the polymers or resins may be for example core/shell; core/shell with shell particles partially encapsulating the core; core/shell particles with a multiplicity of cores; core/shell with a highly cross-linked shell; core/shell with a partially or highly degree of residual unsaturated groups or chemically reactive functional groups; or interpenetrating network particles. The preparation of multistage polymers is described in U.S. Pat. Nos. 3,827,996; 4,325,856; 4,654,397; 4,814,373; 4,916,171; 4,921,898; 5,521,266 and European Pat. No. EP 0 576 128 Al.

The acid monomers provide the requisite ionic strength and base strength responsiveness and the degree of neutralization of the acidic monomers is critical in optimizing the charge density of the acidic groups in both ASE polymers and ASR. The non-ionic vinyl monomers provide an extended polymer backbone structure and added hydrophobic balance. The non-ionic vinyl surfactant monomers provide a bound surfactant. All components contribute to preparing ionic strength and base strength sensitive polymers and barrier compositions whose stability, swell properties and solubility depend on the ionic strength of the aqueous system. Within the stated limits, the proportions of the individual monomers can be varied to achieve optimum properties for specific triggered release applications.

The ASE polymers and ASR require 5-70 weight percent based on total monomer content of one or more acidic monomers selected from the group consisting of C₃-C₈ α,β-ethylenically unsaturated carboxylic acid monomers such as acrylic acid, methacrylic acid, maleic acid, crotonic acid, itaconic acid, fumaric acid, aconitic acid vinyl sulfonic acids and vinyl phosphonic acids, acryloxypropionic acid, methacryloxypropionic acid, monomethyl maleate, monomethyl fumarate, monomethyl itaconate and the like, fatty acids such as lauroleic acid, myristoleic acid, palmitoleic acid, oleic acid, ricinoleic acid, linoleic acid, linolenic acid, eleostearic acid, laconic acid, gadoleic acid, arachidonic acid, erucic acid, clupanodonic acid and nisinic acid, and combinations thereof Acrylic acid (AA), methacrylic acid (MAA) or mixtures thereof and oleic acid are preferred. Mixtures of AA or MAA with itaconic or fumaric acid are suitable and mixyures of crotonic and aconitic acid and half esters of these and other polycarboxylic acids such as maleic acid with C₁-C₄ alkanols are also suitable, particularly if used in minor amount in combination with acrylic or methacrylic acid. For most purposes, it is preferable to have at least about 15 weight percent and most preferably from about 5-50 weight percent of acidic monomers. However, polycarboxylic acid monomers and half esters can be substituted for a portion of the acrylic or methacrylic acid, e.g., about 1-15 weight percent based on total monomer content.

To provide a stable aqueous dispersion and a desirable hydrophobic:hydrophilic balance needed for the ASE polymers and ASR of the present invention requires about 30-95 weight percent of one or more co-polymerizable non-ionic monomers selected from the group consisting of C₂-C₁₈ α,β-ethylenically unsaturated monomers, C₁-C₈ alkyl and C₂-C₈ hydroxy alkyl esters of acrylic and methacrylic acid including ethyl acrylate, ethyl methacrylate, methyl methacrylate, 2-ethylhexyl acrylate, butyl acrylate, butyl methacrylate, 2-hydroxyethyl acrylate, 2-hydroxybutyl methacrylate; styrene, alpha-methyl styrene, vinyltoluene, t-butylstyrene, isopropylstyrene, and p-chlorostyrene; vinyl acetate, vinyl butyrate, vinyl caprolate; acrylonitrile, methacrylonitrile, butadiene, isoprene, vinyl chloride, vinylidene chloride, and the like. In practice, a mono vinyl ester such as methyl acrylate, MMA, ethyl acrylate, butyl acrylate is preferred. In the case of ASR embodiments, mixtures of styrene and mono vinyl esters as well as mixtures of mono vinyl esters are preferred.

These monomers, of course, must be co-polymerizable with the acidic monomers. Normally about 30-95 weight percent, and preferably about 45-95 weight percent of nonionic vinyl monomer, based on total weight of monomers, is used in preparing the polymers.

It has been found that the balance of acidic monomers to non-ionic monomers is an important factor in the triggered release response and performance of the resulting polymers used in barrier or compositions. It is contemplated that the polymers of the present invention have encapsulating properties in addition to having utility as barrier compositions.

Optionally, the polymers include a small amount of at least one polyethylenically unsaturated monomer, to provide a polymer having a network structure. One or more polyethylenically unsaturated monomers may be combined with the monomers during the polymerization process or may be added after the polymerization of monomers. Suitable examples include allyl methacrylate (ALMA), ethylene glycol dimethacrylate (EGDMA), butylene glycol dimethacrylate (BGDMA), diallyl pentaerythritol (DAP), methylenebisacrylamide, pentaerythritol di-, tri- and tetra-acrylates, divinyl benzene, polyethylene glycol diacrylates, bisphenol A diacrylates and combinations thereof. Low levels of the polyethylenically unsaturated monomers are preferred, since levels greater than about 5% by weight tend to over cross-link the polymer or provide a polymer network structure such that their effectiveness in the invention markedly decreases. Preferred amounts of the polyethylenically unsaturated monomers range from 0.001 to 5% by weight based on the total weight of the polymer, more preferably from 0.05 to 1.0% by weight based on the total weight of the polymer.

Another optional monomer component of includes a small amount of at least one metal and/or alkaline earth cross-linking agent, to provide a polymer having a more rigid structure and better mechanical properties. One or more metal and/or alkaline earth cross-linking agents may be combined with the monomers during the polymerization process or may be added after the polymerization of monomers. Suitable metal and/or alkaline earth cross-linking agents include for example alkaline earth ions of calcium, magnesium and barium, transition metal ions of iron, copper and zinc. Other suitable examples such as aluminum ions are described in U.S. Pat. No. 5,319,018. Preferred amounts of the metal and/or alkaline earth cross-linking agents range from 0.01 to 5% by weight based on the total weight of the polymer, more preferably from 0.05 to 5% by weight based on the total weight of the polymer.

In a separate embodiment, there is provided a triggered response composition comprising: one or more polyelectrolytes in contact with an aqueous system that is stable and insoluble in an aqueous system at relatively high ionic strength and that exhibits one or more chemical/physical responses selected from dispersing, degrading, dissolving, destabilizing, deforming, swelling, softening, melting, spreading, flowing and combinations thereof; wherein the chemical/physical response of the composition is triggered upon one or more ionic strength changes, dilution or one or more changes in the concentration of base in the aqueous system. The preferred polymer is an ASE emulsion polymer includes one or more alkali soluble/swellable emulsion polymers comprising: (a) 15-70 weight percent of one or more acidic monomers; (b) 15-80 weight percent of one or more non-ionic vinyl monomers; and optionally (c) 0-5 weight percent of one or more metal cross-linking agents.

In another separate embodiment, there is provided a triggered response composition comprising: one or more polyelectrolytes in contact with an aqueous system that is stable and insoluble in an aqueous system at relatively high ionic strength and that exhibits one or more chemical/physical responses selected from dispersing, degrading, dissolving, destabilizing, deforming, swelling, softening, melting, spreading, flowing and combinations thereof; wherein the chemical/physical response of the composition is triggered upon one or more ionic strength changes, dilution or one or more changes in the concentration of base in the aqueous system. The polyelectrolyte is one or more Morez® polymers comprising: (a) 15-70 weight percent of one or more acidic monomers; (b) 15-80 weight percent of one or more non-ionic vinyl monomers; and optionally (c) 0-5 weight percent of one or more polyethylenically unsaturated monomers or cross-linking. Suitable Morez® polymers and conventional methods used to prepare them are described in U.S. Pat. No. 5,830,957.

In a separate related embodiment employing an ASE emulsion polymer, the composition is a polyelectrolyte of 52.5 weight percent methyl methacrylate (MMA), 29.5 weight percent butyl acrylate (BA), 18 weight percent methacrylic acid (MAA) and 1.5 weight percent 3-mercaptopropionic acid (3-MPA). The polyelectrolyte is stable in an aqueous solution of NaOH of 2.5 M or greater and is triggered to swell/dissolve/disperse by lowering the concentration of NaOH to 1.0 M or less.

In another separate related embodiment employing an ASE emulsion polymer, the composition is a polyelectrolyte of 33 weight percent styrene (Sty), 355 weight percent butyl acrylate (BA), 18 weight percent methyl methacrylate (MMA) and 25 weight percent methacrylic acid (MAA). The polyelectrolyte is stable in an aqueous solution of NaOH of 1.0 M or greater and is triggered to swell/dissolve/disperse by lowering the concentration of NaOH to 0.1 M or less.

The ASE and HASE polymers are conveniently prepared from the above-described monomers by conventional emulsion polymerization at an acid pH lower than about 5.0 using free-radical producing initiators, usually in an amount from 0.01 percent to 3 percent based on the weight of the monomers. The free-radical producing initiators conveniently are peroxygen compounds especially inorganic persulfate compounds such as ammonium persulfate, potassium persulfate, sodium persulfate; peroxides such as hydrogen peroxide; organic hydroperoxides, for example, cumene hydroperoxide, t-butyl hydroperoxide; organic peroxides, for example, benzoyl peroxide, acetyl peroxide, lauroyl peroxide, peracetic acid, and perbenzoic acid (sometimes activated by a water-soluble reducing agent such as ferrous compound or sodium bisulfite); as well as other free-radical producing materials such as 2,2′-azobisisobutyronitrile.

The process for preparing ASE polymers of this invention includes a free radical thermal initiator or redox initiator system under emulsion polymerization conditions. Monomers suitable for the novel process include hydrophobic and hydrophilic monoethylenically unsaturated monomers which can be subjected to free radical polymerization in a straight forward manner. “Hydrophilic” refers to monoethylenically unsaturated monomers which have high water solubility under the conditions of emulsion polymerization, as described in U.S. Pat. No. 4,880,842. “Hydrophobic” refers to monoethylenically unsaturated monomers which have low water solubility under the conditions of emulsion polymerization, as described in U.S. Pat. No. 5,521,266.

The ASE polymers are conveniently prepared from the above-described monomers by conventional emulsion polymerization at an acid pH lower than about 5.0 using free-radical producing initiators, usually in an amount from 0.01 percent to 3 percent based on the weight of the monomers. Alkali swellable/soluble resins are typically prepared by a heated and pressurized reactor (also referred to as a continuous flow tube reactor or Morez® reactor) at temperatures typically less than 3000 C and typically less than 200 psi (kPa) and conventional methods used to prepare them are described in U.S. Pat. No. 5,830,957. Final ASR physical characteristics are dependant upon monomer content, initiator type and quantity, reaction time and reaction temperature.

Free-radical producing initiators including thermal initiators are conveniently employed for preparing HASE, ASE polymers and ASR. Suitable thermal initiators include, for example, hydrogen peroxide, peroxy acid salts, peroxodisulfuric acid and its salts, peroxy ester salts, ammonium and alkali metal peroxide salts, perborate salts and persulfate salts, dibenzoyl peroxide, t-butyl peroxide, lauryl peroxide, 2, 2′-azo bis(isobutyronitrile) (AIBN), alkyl hydroperoxides such as tert-butyl hydroperoxide, tert-amyl hydroperoxide, pinene hydroperoxide and cumyl hydroperoxide, t-butyl peroxyneodecanoate, t-butyl Peroxypivalate and combinations thereof.

Suitable oxidants of the redox initiator system include water-soluble oxidizing compounds such as, for example, hydrogen peroxide, peroxy acid salts, peroxodisulfuric acid and its salts, peroxy ester salts, ammonium and alkali metal peroxide salts, perborate salts and persulfate salts. Suitable oxidants of a redox initiator system also include water-insoluble oxidizing compounds such as, for example, dibenzoyl peroxide, t-butyl peroxide, lauryl peroxide, 2, 2′-azo bis(isobutyronitrile) (AIBN), alkyl hydroperoxides such as tert-butyl hydroperoxide, tert-amyl hydroperoxide, pinene hydroperoxide and cumyl hydroperoxide, t-butyl peroxyneodecanoate, and t-butyl peroxypivalate. Compounds which donate oxygen with free radical formation and are not peroxides, such as alkali metal chlorates and perchlorates, transition metal oxide compounds such as potassium permanganate, managanese dioxide and lead oxide and organic compounds such as iodobenzene, may be usefully employed in accordance with the invention as oxidants. The term “water-insoluble” oxidants means oxidizing compounds having a water solubility of less than 20% by weight in water at 25° C. Peroxides, hydroperoxides and mixtures thereof are preferred and tert-butyl hydroperoxide is most preferred. Typical levels of oxidant range from 0.01% to 3.0%, preferably from 0.02% to 1.0% and more preferably from 0.05% to 0.5% by weight, based on the weight of the monomer used.

Suitable reductants of the redox initiator system include reducing compounds such as, for example, sulfur compounds with a low oxidation state such as sulfites, hydrogen sulfites, alkali metal bisulfites, ketone adducts of bisulfites such as acetone bisulfite, alkali metal disulfites, metabisulfites and its salts, thiosulfates, formaldehyde sulfoxylates and its salts, reducing nitrogen compounds such as hydroxylamine, hydroxylamine hydrosulfate and hydroxylammonium salts, polyamines and reducing sugars such as sorbose, fructose, glucose, lactose and derivatives thereof, enediols such as ascorbic acid and isoascorbic acid, sulfinic acids, hydroxy alkyl sulfinic acids such as hydroxy methyl sulfinic acid and 2-hydroxy-2-sulfinacetic acid and its salts, formadinesulfinic acid and its salts, alkyl sulfinic acids such propyl sulfinic acid and isopropyl sulfinic acid, aryl sulfinic acids such as phenyl sulfinic acid. The term “salts” includes for example sodium, potassium, ammonium and zinc ions. Sodium formaldehyde sulfoxylate, also known as SSF, is preferred. Typical levels of reductant range from 0.01% to 3.0%, preferably from 0.01% to 0.5% and more preferably from 0.025% to 0.25% by weight, based on the weight of the monomer used.

The metal promoter complex of the redox initiator system includes a water-soluble catalytic metal compound in the form of a salt and a chelating ligand. Suitable metal compounds include metal salts such as, for example iron(II, III) salts such as iron sulfate, iron nitrate, iron acetate and iron chloride, cobalt(II) salts, copper(I, II) salts, chromium (II) salts, manganese salts, nickel(II) salts, vanadium salts such as vanadium(III) chloride, vanadium(IV) sulfate and vanadium(V) chloride, molybdenum salts, rhodium salts and cerium(IV) salts. It is preferred that metal compounds are in the form of hydrated metal salts. Typical levels of catalytic metal salts used in accordance with the invention range from 0.01 ppm to 25 ppm. Mixtures of two or more catalytic metal salts may also be usefully employed in accordance with the invention.

Metal complexes that promote the redox cycle in a redox initiator system must not only be soluble, but must have suitable oxidation and reduction potentials. Generally stated, the oxidant must be able to oxidize the low oxidation state of metal promoter complex (e.g. Fe(II)->Fe(III)) and conversely, the reductant must be able to reduce the high oxidation state of the metal promoter catalyst (e.g. Fe(III)->Fe(II)). The choice of a particular oxidant and reductant usefully employed in a redox initiator system for preparing aqueous emulsion polymers from two or more ethylenically unsaturated monomers depends on the redox potentials of the metal salts. In addition, the ratio of oxidant to reductant ranges from 0.1:1.0 to 1.0:0.1, depending on the redox potential of the metal salt employed. For the efficient reduction of monomer levels in an aqueous polymer dispersion prepared from one or more ethylenically unsaturated monomers, it is preferred that the chelating ligand used in combination with the soluble metal salt is a multidentate aminocarboxylate ligand having fewer than six groups available for coordination to the metal salt.

Oxidant and reductant are typically added to the reaction mixture in separate streams or as a single shot, preferably concurrently with the monomer mixture. The reaction temperature is maintained at a temperature lower than 100° C. throughout the course of the reaction. Preferred is a reaction temperature between 30° C. and 85° C., preferably below 60° C. The monomer mixture may be added neat or as an emulsion in water. The monomer mixture may be added in one or more additions or continuously, linearly or not, over the reaction period, or combinations thereof. The type and amount of redox initiator systems may be the same or different in the various stages of the emulsion polymerization.

Optionally, a chain transfer agent and an additional emulsifier can be used. Representative chain transfer agents are carbon tetrachloride, bromoform, bromotrichloromethane, long chain alkyl mercaptans and thioesters such as n-dodecyl mercaptan, t-dodecyl mercaptan, octyl mercaptan, tetradecyl mercaptan, hexadecyl mercaptan, butyl thioglycolate, isooctyl thioglycolate, and dodecyl thioglycolate. The chain transfer agents are used in amounts up to about 10 parts per 100 parts of polymerizable monomers.

Often at least one anionic emulsifier is included in the polymerization charge and one or more of the known nonionic emulsifiers may also be present. Examples of anionic emulsifiers are the alkali metal alkyl aryl sulfonates, the alkali metal alkyl sulfates and the sulfonated alkyl esters. Specific examples of these well-known emulsifiers are sodium dodecylbenzenesulfonate, sodium disecondary-butylnaphthalene sulfonate, sodium lauryl sulfate, disodium dodecyldiphenyl ether disulfonate, disodium n-octadecylsulfosuccinamate and sodium dioctylsulfosuccinate.

Optionally, other ingredients well known in the emulsion polymerization art may be included such as chelating agents, buffering agents, inorganic salts and pH adjusting agents.

Polymerization at an acid pH lower than about 5.0 permits direct preparation of an aqueous colloidal dispersion with relatively high solids content without problems of undue viscosity and coagulant formation. The polymerization is carried out batch-wise, stepwise or continuously with batch and/or continuous addition of the monomers in a conventional manner.

The required monomers can be co-polymerized in such proportions, and the resulting emulsion polymers can be physically blended, to give products with the desired balance of properties for specific applications. Thus, by varying the monomers and their proportions, emulsion polymers having optimum properties for particular triggered response applications can be designed.

In practice it is normally desirable to co-polymerize about 15-60 weight percent based on total monomers, preferably about 20-40 weight percent of one or more acidic monomers, about 15-80 weight percent, preferably about 40-70 weight percent, of one or more non-ionic vinyl monomers and about 1-30 weight percent, preferably about 2-20 weight percent, of one or more non-ionic vinyl surfactant ester monomers. Particularly effective liquid emulsion polymer electrolytes are obtained by copolymerization of a total of about 20-50 weight percent of acrylic acid and methacrylic acid, about 40-70 weight percent of ethyl acrylate, and about 2-12 weight percent of the methacrylic ester of a C₁₂-C₂₄ alkoxypoly(ethyleneoxy) ethyl alcohol.

The synthesis of hydrophobically modified PEL are usefully employed in the present invention. Water-soluble/dispersible/swellable polymers incorporating hydrophobic groups are capable of aggregation and self-organization due to various hydrophobic interactions, such as the non-ionic vinyl surfactant monomer units. If they remain isotropically soluble, such PEL possess an intermediate between homogeneously dissolved PEL and extensively self-organized yet phase-separated systems including for example monolayers and vesicles. PEL with a large number of non-ionic vinyl surfactant moieties linked by a polymer backbone are micelle-forming macromolecules and have utility as triggered response compositions, barrier materials and devices in the invention. Such “micellar” PEL or “polysoaps” have been described in detail by P. Anton, P. Koeberle, and A. Laschewsky in the journal “Makromolekular Chemie”, 194, pp lff, 1993. The synthesis of hydrophobically modified PEL can proceed the synthetic routes including for example modification of preformed macromolecules either by reaction of a hydrophilic polymer with one or more hydrophobic compounds or non-ionic vinyl monomer units or starting with a hydrophobic polymer and introducing hydrophilic moieties, copolymerization of one or more hydrophilic and hydrophobic ethylenically unsaturated monomer units, and polymerization of non-ionic surfactants containing ethylenically unsaturated groups (non-ionic vinyl surfactant monomer units), which affords PEL with the chemically best defined structures. Suitable hydrophilic and hydrophobic polymers are described in U.S. Pat. No. 5,521,266. The combination of polymer and surfactant structures results in several structural architectures that can be modified. This includes for example the length and branching of the polymer side chain, the nature of the ionic “head” group, the nature of the hydrophobic “tail” group, the chemical structure and macromolecular structure of the PEL backbone, and the incorporation of flexible spacer groups, such as PEO units.

Useful compositions related to alkali soluble/swellable polymers and of utility in the present invention include poly(acidic) homopolymers, copolymers and salts thereof. Including for example polycarboxylic acids and salts thereof, polyacrylate salts, HASE, ASE, ASR, Morez® polymers and salts thereof. Suitable examples include Morez® polymers and salts, and combinations thereof. Suitable examples of such polymers are described in U.S. Pat. Nos. 4,095,035; 4,175,975; 4,189,383; 4,267,091; 4,331,572; and 5,830,597. Suitable examples of other polycaboxylic acid polymers also include poly(oxalic acid), poly((meth)acrylic acid), poly(vinyl sulfonic acid), poly(sulfonic acid), poly(sulfuric acid), poly(phosphoric acid), poly(phosphonic acid), poly (vinyl phosphonic acid), poly(maleic acid), poly(beta-malic acid), poly(glutaric acid), poly(fumaric acid), poly(lactic acid), poly(itaconic acid), poly(crotonic acid) and poly(D,L-glutamic acid). PEL of this class are also referred to as anionic PEL.

Anionic, cationic, amphoteric PEL compositions and physical blends or combinations thereof have utility in accordance with the invention as triggered response compositions, barrier materials for encapsulating, and/or surrounding and/or forming a matrix with one or more beneficial agents/active ingredients, and devices for delivering one or more beneficial agents/active ingredients to an environment of use. Environment of use includes for example a liquid medium, an aqueous system, a non-aqueous system, a free flowing solids system, a fabric washing system, a cleaning system, human and animal skin, plant matter. PEL syntheses are optimized to enhance the triggering properties, to enhance the trigger specificity, as well as the activity of the polymers in different triggered response applications and embodiments. Typical examples include alkali swellable/souble polymers, poly(D,L-aspartic) acid, poly(amino acid) polymers, and natural and chemically modified PEL, which incorporate increased ecological and environmental compatability/biodegradability of both PEL and PEL processes. The inventors have provided triggered response PEL including well defined chemical/physical triggers and well defined macromolecular architectures.

Synthetic methods for preparing acid soluble/swellable polymers including emulsion polymers, hydrophobically modified acid soluble/swellable polymers including emulsion polymers, poly(acidic) homopolymers, copolymers and salts thereof; poly(basic) homopolymers, copolymers and salts thereof; amphoteric homopolymers, copolymers and salts thereof including emulsion polymers, poly(amino) acid homopolymers, copolymers and salts thereof; anionic, cationic and amphoteric polysaccharide homopolymers, copolymers and salts thereof; chemically modified anionic, cationic and amphoteric polysaccharides derivatives; anionic, cationic and amphoteric polypeptide homopolymers, copolymers and salts thereof; chemically modified anionic, cationic and amphoteric polypeptide derivatives, chemically modified naturally occurring polypeptides, chemically modified nucleic acids, synthetic nucleic acids, chemically modified enzymes, chemically modified proteins, gelatins and chemically modified gelatins, lignosulfonic acid homopolymers, copolymers and salts thereof; ionene homopolymers, copolymers and salts thereof; anionic, cationic and amphoteric polyester homopolymers, copolymers and salts thereof; chemically modified polyester derivatives both synthetic and naturally occurring; anionic, cationic and amphoteric polyurethane homopolymers, copolymers and salts thereof; chemically modified polyurethane derivatives both synthetic and naturally occurring; copolymer combinations of PEL recited, physical blends of the recited PEL polymers, PEL polymer having cationic, anionic and amphoteric components grafted thereon, is described in “Polyelectrolytes” by H. Dautzenberg, W. Jaeger, J. Koetz, B. Phillip, Ch. Seidel, and D. Stscherbina, Chapters 1-3, Hanser: Munich, 1994; in “Poly(acrylic acid) Thickeners” by R. Y. Lochhead, J. A. Davidson, and G. M. Thomas, in “Polymers in Aqueous Media”, J. E. Glass Ed., ACS: Washington, Chapter 7, 1989; and in “Alkali-Swellable and Alkali-Soluble Thickener Technology” by G. D. Shay, in “Polymers in Aqueous Media”, J. E. Glass Ed., ACS: Washington, Chapter 25, 1989.

Related PEL are cationic polymers and hydrophobically modified cationic polymers. Cationic PEL include for example acid soluble/swellable homopolymers, copolymers and salts thereof including emulsion polymers; hydrophobically modified acid soluble/swellable homopolymers, copolymers and salts thereof including emulsion polymers. Also included are un-neutralized, partially neutralized and completely neutralized PEL as well as un-quaternized, partially quaternized and completely quaternized PEL. Suitable examples of cationic PEL include amine homopolymers, copolymers and salts thereof, quaternized amine polymers, copolymers and salts thereof, poly(amino)acrylates and salts thereof, poly(amido)amines and salts thereof, qauternized poly(amido)amines, poly(acrylate)amines and salts thereof, poly(amino)acrylate esters and salts thereof, polyacrylamides, poly(amino)acrylamides and salts thereof, quaternized poly(amino)acrylamides, poly(amino)urethanes and salts thereof, quaternized poly(amino)urethanes, poly(amino)esters, quaternized poly(amino)esters, poly(acrylate)phosphonates, phosphono-terminated polyacrylates, poly(phosphono)acrylates, poly(sulfonato)acryl ates and salts thereof, polymeric ammonium salts, poly(sulfonium) salts, poly(phosphonium) salts, quaternized poly(amino) alkyl acrylates, copolymers of acid soluble and cationic PEL, physical blends of the recited PEL and cationic PEL salts thereof. Acid soluble and cationic PEL are prepared by conventional solution, suspension and emulsion polymerization. Basic groups such as amino groups and cationic moieties such as quaternary ammonium and phosphonium groups can be prepared by graft polymerization. Blends of acid soluble/swellable and/or cationic PEL homopolymers and copolymers are also usefully employed. Block, alternating and random of acid soluble/swellable and/or cationic PEL copolymers are also usefully employed in the invention. Polymerization conditions such as initiators, temperature, types and kind of ionic and non-ionic monomers as disclosed above for ASE and HASE polymers and as described above are usefully employed.

Polymeric quaternary ammonium containing PEL including ionized and ionizable nitrogen atoms in the polymer backbone are useful in the invention. They are referred in the art as ionenes and afford acid soluble and cationic PEL.

Cationic PEL also having utility are prepared from the chemical modification of polyacrylamides by the following reactions including for example base catalyzed Mannich reaction of formaldehyde and alkyl amines with polyacrylamides, reaction of polyacrylamides with an amine containing a primary and a tertiary function leading to a amino-substituted PEL with pendant tertiary amine groups, and Hofmann reaction on polyacryamides using for example basic hypochlorite resulting in polyvinyl amino PEL. The former results in stable PEL by subsequent quaternization of the amine function. Polyacrylonitriles are usefully chemically modified in a similar manner.

The acid soluble and cationic PEL require 15-70 weight percent based on total monomer content of one or more basic and cationic monomers selected from the group consisting of C₃-C₈ α,β-ethylenically unsaturated amino monomers such as N-alkyl (amino)acrylates, N-alkyl (amino)methacrylic acid, N,N-dialkyl(amino) acrylates and methacrylates, (amino)acrylamides and methacrylamides, N-alkyl acrylamides, (vinyl)amino sulfonates and vinyl phosphonates, N-substituted (ammonium) acrylates and (ammonium) alkyl acrylates, (phoshonium) acrylates, terminally substituted phosphonium acrylates and combinations thereof. Other suitable acid soluble and cationic monomers include for example diallyldimethylammonium halides (e.g. chloride is referred to as DADMC), dimethylaminoethylacrylate and methacrylate, dimethylaminopropylmethacrylate, dimethylaminomethacrylamide, acryoxyethyltrimethylammonium halides, methacrylamidopropyltrimethyl ammonium halides, 3-methacryloxy(2-hydroxy)propyltrimethylammonium halides, and (3-acrylamido-3-methyl)butyltrimethylammonium halides and combinations thereof. Half esters of these and other polyethylenically unsaturated amines and polyvinyl amines with maleic acid with C₁-C₄ alkanols are also suitable. For most purposes, it is preferable to have at least about 15 weight percent and most preferably from about 20-50 weight percent of basic and cationic monomers. Acid soluble/swellable emulsion polymers, hydrophobically modified acid soluble/swellable emulsion polymers can be converted to cationic and hydrophobically modified PEL using conventional acids and alkylation reactions. Cationic quaternary ammonium monomers derived from AA and MAA and their homopolymers as well as their copolymers with acrylamide are useful because of their utility in manifold applications. Monomeric N-substituted acrylamides are more expensive than N-akylaminoacrylates, but the former offer several advantages and utility including a higher reactivity of monomer units and a comparatively increased hydrolytic stability of both the monomer and PEL. Copolymers of cationic monomers such as DADMAC and one or more ethylenically unsaturated monomers including for example acrylonitrile, methylstearyldiallylammonium chloride, vinyl acetate, styrene, alkyl acrylates, AA, MAA, and maleic anhydride are usefully employed in the invention. Suitable poly(amines) including poly(D, L-lysine) and poly(amideamine) are also usefully employed in the invention. Copolymers of acrylamide and DADMAC are also useful.

Copolymerization of cationic vinyl monomers with non-ionic co-monomer usefully provides PEL with variable charge density, charge strength and degrees of neutralization. Charge density can be verified by reaction of different amounts of both co-monomers in the initial co-monomer mixture or in the feed. PEL having different charge strength can be obtained using alkyl(amino) and quaternary ammonium derivatives of AA and MAA as recited above. Polymeric cationic PEL containing a pendant aromatic nucleus are useful in the invention and are obtained by polymerization of vinyl monomers including for example alkylamino styrene, (p-vinyl(benzyl) triakylammonium halides), vinylpyrines, vinylpyridinium halides, pyrollidones and vinylpyrollidinium halides. Polymerization in aqueous solution requires a low pH to ensure polymer and emulsion stability, in which case the nature of the charges in the cationic PEL changes considerably by virtue of controlled partial ionization. Basic, vinyl heterocyclic monomers are also usefully employed including for example vinyl imidazole, vinyl imidazolinium, vinyl piperdine and vinyl piperdinium halides.

Useful compositions related to acid soluble/swellable polymers and of utility in the present invention are basic homopolymers, copolymers and salts thereof. Suitable example include ammonium and quaternary ammonium salts of polyamines and poly(amino)acrylates, alkyl ammonium salts of polyamines and poly(amino)acrylates, phosphonium salts of polyamines and poly(amino)acrylates, sulfonium salts of polyamines and poly(amino)acrylates, and combinations thereof.

Amphoteric PEL are usefully prepared by free radical polymerization. The presence of both anionic and cationic charges has a distinct effect on the solution state and solid state properties of these PEL. The hydrodynamic volume of an amphoteric PEL are effected by aqueous system parameters including for example pH, charge density, salt concentration, ionic strength, types and concentrations of added salts and combinations thereof. In the absence of low molecular weight PEL a large number of PEL is not soluble in aqueous media but exists as hydrogels. The extent of such effects can be modified by incorporating one or more non-ionic monomers in to the growing PEL polymer chain. The inventors have discovered that the polymerization process are influenced by such parameters in the aqueous system. Synthesis of amphoteric PEL by free radical polymerization includes for example copolymerization of acidic and basic ethylenically unsaturated monomer units, such as acidic and basic monomer units including for example AA and alkyl(amino) acrylates. Variation of ionic strength and pH results in changes in reactivity of the ionizable monomer units, for example with unionized AA and the carboxylate ion. The classical two component copolymer is not applicable in such an instance. Polymerization of amphoteric ion-pair comonomers in solution, suspension or emulsion is also useful in the invention. Such amphoteric monomers include for example vinyl anionic monomers, which are the gegenions (counter-ions) of a vinylic cationic monomer units. Non-polymerizable ions are absent. The monomer pair is isolated and characterized. Polymerization of such ion pairs is described as a homopolymerization of a monomer incorporating two individually polymerizable ethylenically unsaturated groups by J. C. Salmone, C. C. Tsai, A. C. Watterson, and A. P. Olson in “Polymeric Amines and Ammonium Salts”, Ed.: E. Goethals, Pergamon Press: New York, pp. 105 ff, 1980. the resulting PEL bulk includes equimolar amounts of cationic and anionic charges pendant along the polymer chains. The distribution of charges over the PEL is random, since the incorporated polymerized comonomers are not alternating, and additionally, not every individual polymer chain contains necessarily an equal amount of cationic and anionic monomer units. Optionally, terpolymerization of ion pair commoners with one or more non-ionic monomer units affords amphoteric PEL ionomers with enhanced rigidity by the presence of ionic interactions. Polydispersities and molecular weights depend on any solvent which affects the degree of intermolecular aggregation. Also useful for the synthesis of amphoteric PEL are polymerization of sulfobetaine and carbobetaine monomer units. The resulting PEL have a well defined arrangement of ionic charges. The zwitterions in such PEL remain in their di-ionic form over a broader range of ionic strength and pH. Each monomer unit includes both anionic and cationic sites on the same pendant group and are readily polymerizable in aqueous systems. Such PEL tend to exhibit a hydrogel character, as evidenced by the inter- and intramolecular ionic interactions of the cationic and anionic charges. Additions of simple salts promotes water solubility/dispersity of the PEL. In contrast to the behavior of other PEL, the viscosity of the aqueous system of polymeric zwitterions increases with increasing salt concentration.

Amphoteric PEL are usefully employed in the present invention. Suitable example include are poly(amino)acids such as poly(D,L-aspartic acid), poly(glycine) and (D, L-phenyl alanine). A useful method for preparing such poly(amino)acids are the chemical modification of homopolymers and copolymers including for example aminolysis of alternating copolymers of maleic anhydride with excess diamines, affording regular polyamphoteric PEL containing amine and carboxylic groups, hydrolysis of cyclic polymers containing amide bonds in the ring, which can be readily prepared by cyclopolymerization, resulting in polyamphoetric PEL, and interactions of neighboring functional groups during Curtius-, Lossen-, Hofmann-type rearrangements on preformed polymers leading to amphoteric PEL of regular, alternating sequences, exemplified by the Hofmann degradation of polyacrylonitrile, providing a simple route to a random copolymer of AA and vinyl amine. In addition, for example, reaction of polyacrylonitrile with dicyandiamide as well as with hydroxylamine affords amphoteric PEL, which are soluble/dispersible only in acidic or basic media and high ionic strength or low ionic strength media. Between pH 3 and 9 they are insoluble in aqueous systems, forming sedimenting flocs. Such PEL have utility as for example flocculants, sequestering agents for active ingredients, encapsulation of beneficial agents and immobilization agents.

Useful acidic, basic, cationic and anionic monomers usefully employed in the invention for preparing amphoteric PEL are described above. In addition, suitable monomer units for preparing such PEL copolymers include for example allylic and diallylamino monomers with MA and maleamic acids. Such PEL have regular alternating structures. The pH of the reaction mixture of such monomers have values corresponding to the respective isoelectric points of the resulting PEL.

Both synthetic and natural PEL are usefully employed in the present invention. Suitable natural polymers for preparing such PEL include for example polysaccharides, polysaccharide derivatives, proteins, nucleic acids and lignin. Depending on the staring natural polymers and the PEL macromolecular structure intended, PEL are obtained from such biological polymer (“biopolymers”) by synthetic methods including for example isolation of a preformed PEL from the moiety (monomer unit motif) of the natural product by conventional extraction and precipitation techniques, isolation by a combination of extraction and chemical modification in order to liberate a preformed ionogenic group and/or to degrade the natural product for obtaining a soluble/swellable/dispersible PEL and derivatization of an isolated non-ionic polymer to an anionic, cationic or amphoteric PEL.

Suitable examples of amphoteric natural PEL include for example polyesters of the integral type composed of phosphoric acid and deoxyribose units, respectively, with a heterocyclic weak base attached to the carbohydrate unit, also referred to as nucleic acids. In aqueous systems, these nucleic acids usually behave as an anionic PEL with Na+ ions acting as counter-ions to the phosphoric acid units with one relatively strong acid function. The variability of nucleic acid PEL macromolecular structures includes for example the choice of the type and sequence of heterocyclic weak N-bases adenine, guanine, thymine, cytosine, cysteine and uracil attached to sugar moiety of the biopolymer backbone, the choice of sugar unit, namely ribose in the case of ribonucleic acids (RNA) and deoxyribose in the case of deoxyribonucleic acids (DNA) and the biopolymer chain conformation stabilized by hydrogen bonding (H-bonding) originating from the attached heterocyclic bases to the sugar moieties.

Related to nucleic acids are teichoic acids which are also included. Teichoic acids are linear polyesters composed of phosphoric acid units and glycerol, respectively, ribitol units reacting as a diol and carrying various sugar and amino acid constituents as side groups. The anionic character of these water-soluble/swellable/dispersible PEL results from the free acid function of the phosphoric acid units not involved in ester linkages, analogous to nucleic acids. Teichoic acids are found in a variety of microorganisms including, for example, Lactobacillus cerabiosus and can be isolated from them by conventional techniques.

Additional suitable natural PEL usefully employed in the invention are polypeptide and protein based PEL homopolymers, copolymers and salts thereof, and chemically modified derivatives of natural polypeptides and proteins. The monomer units of such biopolymers are α-amino carbonic acids of the general formula RCHNH₂.COOH which are linked via peptide bonds, namely, amide linkages between the amino and the adjacent carboxylic group. Anionic, cationic and amphoteric polyelectrolytic peptides and proteins are obtained, especially if the monomer contains additional acidic and basic functional groups. Suitable examples of amphoteric PEL usefully employed in the present include poly(aminocarboxylic acids) such as poly(D, L-aspartic acid), poly(glycine), poly(D, L-phenyl alanine), type-A gelatins, type-B gelatins and collagens. The synthesis of polyaspartic acid is described in detail in U.S. Pat. Nos. 5,057,597; 5,328,631; 5,319,145; 5,491,212; 5,380,817; 5,484,878; 5,371,170; 5,410,017; 5,459,234; 5,457,176; 5,552,514; 5,556,938; 5,554,721; 5,658,464; 5,531,934 and European Pat. Nos. EP 0 700 987; EP 0 705,794; EP 0 644 257; and EP 0 625 531.

Additional suitable natural PEL usefully employed in the invention are polysaccharide-based PEL homopolymers, copolymers and salts thereof and chemically modified derivatives. Most of natural polymer based PEL have a polysaccharide backbone, with the ionic group being chemically attached as side groups and the PEL representing the pendant type. Suitable polysaccharide-based PEL include for example cycodextrins, glucoses, pentoses, hexoses, glucosidic derivatives (half acetals), celluloses, chemically modified celluloses, cellulose derivatives, microcrystalline celluloses, galactoses, starches, mannoses, lactoses, fructoses, sucroses, gel forming anionic galactans such as carrageenans, carrageenan fractions, agars such as agarose, chemically modified agaroses, D-galactose and agaropectin, pectins such as poly-D-galacturonic acid and its esters, gel forming anionic galactans containing sulfate half-ester groups, such as derived from marine algaes, furcellans, porphyrans, phyllophyran, and ascophyllan, aligns, alginic acids, mannuronic acids, guluronic acids, alginate salts, traganth, traganth gums having arabinose, galactose, fucose and xylose units, gum arabic, hylauronic acids such as D-glucuronic acid, PEL obtained fromnatural polymer products by liberation of preformed ionic sites such as pectins or chitosans, and heparins.

Polysaccharide-based PEL are mostly anionic in character and their respective macromolecular structure linear, branched, block copolymers, and blends of saccharides and other polymers. The anionic PEL are due to carboxylate and sulfate half-ester groups attached to side chains or the polymer backbone. They may also be obtained plant tissue, animal tissue, plant extracts, animal extracts, microbial products and chitin, bone, cartilage, and cellular extracts. Cellulose-based PEL are a subclass of PEL that have utility in the present invention. Such PEL are conventionally prepared by synthetic methods including for example a two-phase system with cellulose as at least initially solid phase, esterification of cellulose affording anionic polyelectrolytic esters such as cellulose xanthogenate and cellulose phosphate esters, etherification of cellulose to afford PEL such as carboxymethylcellulose (CMC), carboxymethylcellulose, dicarboxymethyl cellulose, and sulfoethyl cellulose, epoxidation of cellulose, aminoalkylation of cellulose, oxidation of cellulose to afford PEL such as 6-carboxycellulose, anhydroglucose. Xylan-based PEL are a subclass of PEL that have utility in the present invention. Starch-based PEL are a subclass of PEL that have utility in the present invention. Suitable examples include anionic starch esters such as starch phosphates, anionic ethers, and cationic starches. Dextran-based PEL are a subclass of PEL that have utility in the present invention. Lignin-based PEL derived from wood and wood products are a cellulose related class of cross-linked PEL that have utility in the present invention.

PEL cannot be understood as a simple superposition of electrolyte and polymer properties. Whereas excluded volume effects are the only important interaction in non-ionic polymers, the long range Coulomb interactions in PEL gives rise to a wide variety of trigger means in aqueous systems. In contrast to simple electrolytes, one type of charge is bundled together along a polymer chain, resulting in strong fields near the polymer chain even at high dilution in aqueous systems. This unique feature of PEL is useful for manipulating the ionic strength of a liquid medium to create various ionic triggers and is believed to be responsible for PEL exhibiting rod-like behavior in aqueous systems at infinite dilution and without added salts. Useful electrochemical properties of PEL are determined by the content and state of dissociation of the ionized and/or ionizable groups of the ionic macromolecules which provide useful trigger means in aqueous systems based on the following parameters including for example potentiometric triggers in the presence or absence of added salts, the degree of dissociation as a function of ionic strength (equilibria), structural triggers based on potentiometric changes, effects of added polyanions and buffers, triggers based on conductance changes, ionic strength and salt concentration dependence on conductance triggers, electrophoretic triggers based on changes in ion mobility on both macroscopic and microscope domains, adsorption triggers, Ultraviolet (UV) and visible triggers based on changes in radiation responsive functions and certain chromophores incorporated in the monomeric units of the PEL, luminescence triggers, UV and visible light triggers and fluorescence triggers.

In general, the ASE and HASE copolymer dispersions obtained have a solids content ranging from 20 to 50% by weight and the copolymer has a weight average molecular weight of about 200,000 to 10,000,000, when no polethylenically unsaturated monomer or metal cross-linking agent is incorporated into the polymer, as determined by gel permeation chromatography (GPC). A chain transfer agent may be used to obtain weight average molecular weights down to 30,000 or lower.

The HASE copolymer products prepared by emulsion polymerization at an acid pH are in the form of stable aqueous colloidal dispersions usually with a typical milky latex appearance. Such a liquid emulsion contains the copolymer dispersed as discrete particles having average particle diameters of about 500-300000 Å, as measured by light scattering.

In the form of a stable, aqueous colloidal dispersion at an acid pH of about 2.5-5.0 the ASE and HASE copolymers are particularly useful and have desirable film forming properties. Such aqueous dispersion may contain about 10-50 weight percent of polymer solids yet be of relatively low viscosity. Thus it is readily metered and blended with aqueous product systems. However, the dispersion is ionic strength and/or pH responsive. When the ionic strength and/or pH of the polymer dispersion is adjusted by addition of a base such as ammonia, an amine or a non-volatile inorganic base such as sodium hydroxide, potassium carbonate or the like, the aqueous mixture becomes translucent or transparent as the polymer dissolves at least partially in the aqueous phase with a concurrent increase in viscosity. This neutralization can occur in situ when the liquid emulsion polymer is blended with an aqueous solution containing a suitable base. Or if desired for a given application, pH adjustment by partial or complete neutralization can be carried out before or after blending the liquid emulsion polymer with an aqueous product.

The ASE copolymer dispersions obtained have a solids content ranging from 20 to 50% by weight and the ASE copolymer has a weight average molecular weight of about 200,000 to 10,000,000, when no polyethylenically unsaturated monomer or metal cross-linking agent is incorporated in to the polymer, as determined by gel permeation chromatography (GPC). A chain transfer agent may be used to obtain weight average molecular weights down to 30,000 or lower. The ASR aqueous dispersions obtained have a solids content ranging from 10 to 50% by weight and the ASR has a weight average molecular weight of from 1,000 to 20,000 when no polyethylenically unsaturated monomer or metal cross-linking agent is incorporated in to the polymer, as determined by gel permeation chromatography (GPC). Typical pH of ASR aqueous ammonia dispersions are between 7.0 to 9.0. ASR dispersion at an acidic pH are in the form of stable colloidal dispersions with a typical opaque appearance. Typical viscosities of ASR range between 300 and 2500 cps and have been 25 to 35% by weight non-volatiles. The Morez® polymers typically are prepared in the form of resins or a prepared as ammonia neutralized aqueous solutions. Such a liquid dispersion contains the copolymer dispersed as discrete particles having average particle diameters of about 5-3000 Å, as measured by light scattering. Particle size can range between 0.5 nm to 3000 μm depending on polymerization conditions and processes employed.

The ASE copolymer products prepared by emulsion polymerization at an acid pH are in the form of stable aqueous colloidal dispersions usually with a typical milky latex appearance. Such a liquid emulsion contains the copolymer dispersed as discrete particles having average particle diameters of about 500-3000 Å, as measured by light scattering. Particle size can range between 5 nm to 3000 μm depending on polymerization conditions and processes employed.

In the form of a stable, aqueous colloidal dispersion at an acid pH of about 2.5-5.0 both the ASE copolymers and ASR are particularly useful in preparing barrier materials and have desirable film forming properties. Such aqueous dispersion contain about 10-50 weight percent of polymer solids yet are of relatively low viscosity. Thus it is readily metered and blended with aqueous product systems. However, the dispersion is responsive to changes in base strength, pH, ionic strength and/or to dilution of the aqueous system. When the ionic strength and/or pH of the polymer dispersion is adjusted by addition of a base such as ammonia, an amine or a non-volatile inorganic base such as sodium hydroxide, potassium carbonate or the like, the aqueous mixture becomes translucent or transparent as the polymer dissolves at least partially in the aqueous phase with a concurrent increase in viscosity. This neutralization can occur in-situ when the liquid emulsion polymer is blended with an aqueous solution containing a suitable base. Or if desired for a given application, pH adjustment by partial or complete neutralization or no pH adjustment can be carried out before or after blending the liquid emulsion polymer with an aqueous product.

The glass transition temperature (“Tg”) of the ASE and HASE polymers typically range from −60° C. to 150° C., preferably from −20 C to 50° C., the monomers and amounts of the monomers selected to achieve the desired polymer Tg range are well known in the art. Tgs used herein are those calculated by using the Fox equation (T.G. Fox, Bull. Am. Physics Soc., Volume 1, Issue No. 3, page 123(1956)). that is, for calculating the Tg of a copolymer of monomers M1 and M2,

1/Tg(calc.)=w(M1)/Tg(Ml)+w(M2)/Tg(M2), wherein

Tg(calc.) is the glass transition temperature calculated for the copolymer

w(M1) is the weight fraction of monomer M1 in the copolymer

w(M2) is the weight fraction of monomer M2 in the copolymer

Tg(M1) is the glass transition temperature of the homopolymer of M

Tg(M2) is the glass transition temperature of the homopolymer of M2,

All temperatures being in ° K. The glass transition temperatures of homopolymers may be found, for example, in “Polymer Handbook”, edited by J. Brandrup and E. H. Immergut, Interscience Publishers.

The term “liquid emulsion polymer” as applied to the ASE and HASE polymers means the polymer was prepared by emulsion polymerization even though the polymer per se may be (and generally is) a solid at room temperature but is a “liquid” emulsion polymer because it is in the form of a liquid solution or dispersion.

In a preferred embodiment of the invention, ASE and HASE polymers of are advantageous for use as barrier compositions that surround or encapsulate one or more active ingredients/beneficial agents. Two or more ASE and/or HASE polymers may be used, if desired. Of course the HASE polymers are preferably film-forming at temperatures below about 25° C., either inherently or through the use of plasticizers. It has been discovered that both ASE and HASE polymers form effective barrier materials for surrounding and/or encapsulating one or more active ingredients immersed in an aqueous system, such that the stability of the barrier materials changes by altering the ionic strength, pH, temperature, mechanical forces and the combinations thereof the aqueous system. In an aqueous system the materials are stable, forming effective barriers to contain or encapsulate one or more actives. Exposing the materials to a subsequent aqueous system triggers instability in the materials such that the active ingredients are rapidly dispersed in the aqueous system.

In a preferred embodiment, barrier compositions prepared from one or more ASE and/or HASE polymers from impermeable membranes that surround or encapsulate one or more active ingredients, providing sufficient structural support while inhibiting the release of the beneficial agent prior to the ionic strength triggered dissolution of the barrier of the device. Aqueous system refers to any fluid or solution containing water as the principal liquid component (e.g. solutions of organic or inorganic substances particularly electrolytes, mixtures of substance in water and physiological fluids). Typically the barrier composition totally surrounds, encapsulates and/or forms a matrix with the beneficial agent/active ingredient. One or more additives may be combined with the ASE and HASE polymers to prepare a composite barrier to totally surround, encapsulate and/or form a matrix with the beneficial agent if desired. The barrier and composite barrier materials have a combination of thickness and mechanical strength so that they are disrupted by the triggered response of the ASE and HASE polymers (triggered response compositions) thus releasing the beneficial agent. Preferably the barriers are 0.1 μm to 1 mm in thickness. Preferably the barriers are 10 μm to 300 μm in thickness for personal care and cleaning applications. The barrier may be a thin film, a dense film, a composite barrier, a container, a capsule, and matrix beads.

Typically, a barrier composite is composed of the triggered response polymers and polymers, biopolymers, and any other naturally occurring and synthetic material, although appropriately treated inorganic materials such as ceramics, metals or glasses may be used. The following is a preferred listing of components and additives that can be incorporated into the barrier material and device of the present invention.

Cellulose esters such as cellulose acetate, cellulose acetate acetoacetate, cellulose acetate benzoate, cellulose acetate butylsulfonate, cellulose acetate butyrate, cellulose acetate butyrate sulfate, cellulose acetate butyrate valerate, cellulose acetate caprate, cellulose acetate caproate, cellulose acetate caprylate, cellulose acetate carboxymethoxypropionate, cellulose acetate chloroacetate, cellulose acetate dimethaminoacetate, cellulose acetate dimethylaminoacetate, cellulose acetate dimethylsulfamate, cellulose acetate dipalmitate, cellulose acetate dipropylsulfamate, cellulose acetate ethoxyacetate, cellulose acetate ethyl carbamate, cellulose acetate ethyl carbonate, cellulose acetate ethyl oxalate, cellulose acetate furoate, cellulose acetate heptanoate, cellulose acetate heptylate, cellulose acetate isobutyrate, cellulose acetate laurate, cellulose acetate methacrylate, cellulose acetate methoxyacetate, cellulose acetate methylcarbamate, cellulose acetate methylsulfonate, cellulose acetate myristate, cellulose acetate octanoate, cellulose acetate palmitate, cellulose acetate phthalate, cellulose acetate propionate, cellulose acetate propionate sulfate, cellulose acetate propionate valerate, cellulose acetate p-toluene sulfonate, cellulose acetate succinate, cellulose acetate sulfate, cellulose acetate trimellitate, cellulose acetate tripropionate, cellulose acetate valerate, cellulose benzoate, cellulose butyrate napthylate, cellulose butyrate, cellulose chlorobenzoate, cellulose cyanoacetates, cellulose dicaprylate, cellulose dioctanoate, cellulose dipentanate, cellulose dipentanlate, cellulose formate, cellulose methacrylates, cellulose methoxybenzoate, cellulose nitrate, cellulose nitrobenzoate, cellulose phosphate (sodium salt), cellulose phosphinates, cellulose phosphites, cellulose phosphonates, cellulose propionate, cellulose propionate crotonate, cellulose propionate isobutyrate, cellulose propionate succinate, cellulose stearate, cellulose sulfate (sodium salt), cellulose triacetate, cellulose tricaprylate, cellulose triformate, cellulose triheptanoate, cellulose triheptylate, cellulose trilaurate, cellulose trimyristate, cellulose trinitrate, cellulose trioctanoate, cellulose tripalmitate, cellulose tripropionate, cellulose trisuccinate, cellulose trivalerate, cellulose valerate palmitate and combinations thereof Cellulose ethers such as 2-hydroxybutyl methyl cellulose, 2-hydroxyethyl cellulose, 2-hydroxyethyl ethyl cellulose, 2-hydroxyethyl methyl cellulose, 2-hydroxypropyl cellulose, 2-hydroxypropyl methyl cellulose, dimethoxyethyl cellulose acetate, ethyl 2-hydroxylethyl cellulose, ethyl cellulose, ethyl cellulose sulfate, ethylcellulose dimethylsulfamate, methyl cellulose, methyl cellulose acetate, methylcyanoethyl cellulose, sodium carboxymethyl 2-hydroxyethyl cellulose, sodium carboxymethyl cellulose. Polycarbonates. Polyurethanes. Polyvinyl acetates. Polyvinyl alcohols. Polyesters. Polysiloxanes such as poly(dimethylsiloxane) and Polyaminoacids such as polyaspartic acid. Polyacrylic acid derivatives such as polyacrylates, polymethyl methacrylate, poly(acrylic acid) higher alkyl esters, poly(ethylmethacrylate), poly(hexadecyl methacrylate-co-methylmethacrylate), poly(methylacrylate-co-styrene), poly(n-butyl methacrylate), poly(n-butyl-acrylate), poly(cyclododecyl acrylate), poly(benzyl acrylate), poly(butylacrylate), poly(secbutylacrylate), poly(hexyl acrylate), poly(octyl acrylate), poly(decyl acrylate), poly(dodecyl acrylate), poly(2-methyl butyl acrylate), poly(adamantyl methacrylate), poly(benzyl methacrylate), poly(butyl methacrylate), poly(2-ethylhexyl methacrylate), poly(octyl methacrylate), acrylic resins. Polyethers such as poly(octyloxyethylene), poly(oxyphenylethylene), poly(oxypropylene), poly(pentyloxyethylene), poly(phenoxy styrene), poly(secbutroxylethylene), poly(tert-butoxyethylene), copolymers thereof and polymer blends thereof.

Typical naturally occurring materials include: insect and animal waxes such as chinese insect wax, beeswax, spermaceti, fats and wool wax; vegetable waxes such as bamboo leaf wax, candelilla wax, carnauba wax, Japan wax, ouricury wax, Jojoba wax, bayberry wax, Douglas-Fir wax, cotton wax, cranberry wax, cape berry wax, rice-bran wax, castor wax, indian corn wax, hydrogenated vegetable oils (e.g., castor, palm, cottonseed, soybean), sorghum grain wax, Spanish moss wax, sugarcane wax, caranda wax, bleached wax, Esparto wax, flax wax, Madagascar wax, orange peel wax, shellac wax, sisal hemp wax and rice wax; mineral waxes such as Montan wax, peat waxes, petroleum wax, petroleum ceresin, ozokerite wax, microcrystalline wax and paraffins; and synthetic waxes such as polyethylene wax, Fischer-Tropsch wax, chemically modified hydrocarbon waxes including polyethyleneglycolated waxes and cetyl esters wax.

In a preferred embodiment, the ionic strength trigger is an ionic strength sensitive barrier composition surrounding the ingredients, the barrier substantially impermeable to releasing the active ingredients to the aqueous system and remaining insoluble in the aqueous system at relatively high ionic strength (for example, equivalent to 0.01 M sodium carbonate or greater), the barrier becoming soluble in an aqueous system at relatively lower ionic strength (for example, equivalent to less than 0.01 M sodium carbonate) and effecting the rapid release of the active ingredients.

The triggered response composition in the barrier material or the device is usefully employed in the invention in form of, for example, polymer particles, a film, a coating, a tablet, capsule, pellet, sachet, matrix beads, and encapsulated polymer granules or supported on a substrate. Suitable substrates include for example films, non-woven textiles, woven textiles, solids, paper, fabric, and skin. The ionic strength responsive trigger means is provided in a capsule or tablet by for example bonding, encasing, friction fitting, partially encasing the barrier material, for example, either as an adhesive, joining portions of the barrier, as an outer coating, or forming encapsulated particles and co-granulated particles together to form the capsule or tablet. The ionic strength responsive trigger means in the aqueous system causes bursting of the device followed by release of one or more beneficial agents/active ingredients.

Optionally, the ionic strength responsive barrier materials are trigger response polymer blends or they are blended with an inert non-dissolving material. By inert is meant a material that is not substantially affected by a change in ionic strength and/or pH in the triggering range. By altering the proportion of a ionic strength and pH-responsive material to one or more inert non-dissolving materials, the time lag subsequent to triggering and prior to release may be controlled. The inert non-dissolving material is added to further provide mechanical strength and stability to the barrier material or device during use (for example, after the polymer and barrier swells) or storage. Typical inert non-dissolving material usefully employed in the invention is listed the materials described as additives to the barrier material or device. Preferably, the inert material is selected from the list of additives given above.

The term beneficial agent refers to substances for which it is desirable and/or advantageous to triggered delivery into an environment of use. Beneficial agents include those agents in the form of a gas, solid or liquid state.

The term beneficial agent refers to substances for which it is desirable and/or advantageous to control delivery into an environment of use. Examples of such substances include: detergent additives and cleaning additives including, for example, fabric softeners, fabric softener formulations, cationic and anionic surfactants, scale controllers, anti-foaming agents, buffers, amphoteric additives, builders, bleaches, organic additives, inorganic additives, whiteners, dyestuffs, stain removers, water hardness agents, reductants, oxidants, optical brighteners, UV protective agents, wrinkle reducing agents, gray-inhibitors, soil repellants, oil-absorbing polymers, waterproofing polymers, active-retaining polymers, redeposition agents, anti-redeposition agents, polymers which inhibit the formation of soil and oily materials, detergent additive formulations, biocidal compositions and formulations, antimicrobial compositions and formulations, activating agents, stabilizing agents, polymers with special detergent properties such as co-builders and anti-redeposition agents, pH controlling agents, enzymes, enzyme inhibitors, disinfectants, personal care agents, water softening agents, absorbants, flavor, fragrances, personal care actives and pharmaceutically effective agents. Suitable examples of pharmaceutically effective agents/beneficial agents are described in U.S. Pat. No. 5,358,502.

Although any mixture of the above ingredients may be used that satisfactorily delivers the beneficial agent, typically the ionic strength-trigger means is 0.01% to 50% by weight of the device and the barrier including ionic strength-trigger means is typically 1% to 30% of the device. Preferably the ionic strength-trigger means is 0.1% to 20% of the device and the membrane, including ionic strength-trigger means, is 1% to 20% of the device. The amount of beneficial agent is the amount that is sufficient to achieve the desired effect (e.g., cleaning effect, softening effect personal care effect, and combinations thereof). The remainder weight can be made up of any desired formulation ingredients (described above) and other additives.

The devices of the invention preferably contain a solid beneficial core or a liquid beneficial core. Optionally, the devices of this invention can also be administered within a capsule comprising a water-soluble wall. For example, the devices can be manufactured to be of suitable size for inclusion either singularly or multiply within a gelatin capsule such that when the capsule dissolves the device(s) are released into the environment of use. While the devices to be included within a capsule can be of a variety of shapes, a preferred shape for such devices is spherical or substantially spherical. The exact number and size of such devices can and will be determined according to a variety of well known factors. For example, the environment of use, the beneficial agent or agents, the amount of beneficial agent and the rate of release are all factors to be considered in determining the size, shape, and number of devices to be included in such capsules as well as the composition of the capsule.

The devices of this invention having the above described desired characteristics may be made using the above described materials using the following processes and other conventional methods.

Capsule formulations may be prepared by forming a cap and body of the above-described polymers. In a conventional fashion, the triggered response polymers may be molded into the desired shapes and sintered or dip-coated (in a similar fashion to the way hard gelatin capsules are made). Preferably they are by conventional coating techniques including, for example, spray coating, wurster coating and pan coating. Alternatively, hard gelatin capsules may be coated with the barrier coating. These capsule bodies and caps are then filled with the beneficial agent in the form of a gas, liquid or solid and other excipients (e.g., osmagent, swellable component) using standard capsule filling techniques. Then the capsule is sealed with the desired ionic strength-responsive material and assembled. This may be performed using conventional capsule-sealing equipment.

Tablets may be prepared using conventional processes and conventional tableting and tablet-coating equipment. The tablet cores can be made by direct compression of the beneficial agent and other desired excipients (e.g., osmagent swellable material) or other common tableting methods. To minimize incompatibilities or provide a suitable substrate for the barrier coating, the tablets may first be coated with a water-soluble pre-coat. The pre-coat may consist of sugars, salts, soluble cellulose derivatives or other water-soluble materials.

The tablet cores are coated with either a dense triggered response barrier material or composite using conventional coating techniques. These films can be applied using conventional equipment such as fluid-bed coaters, pan-coaters, Wurster coaters, spray-dryers or by dip-coating.

In one preferred embodiment, the barrier composition is stable and insoluble in an aqueous system at relatively high ionic strength; wherein the barrier exhibits one or more chemical/physical responses selected from dispersing, disintegrating, dissolving, destabilizing, deforming, swelling, softening, flowing and combinations thereof, wherein the chemical/physical response of the composition is triggered upon one or more ionic strength changes to the aqueous system; wherein the device is capable of releasing the active ingredients to the aqueous system as a result of the triggered response of the barrier composition; wherein the device is prepared using coating technology selected from the group consisting of fluid bed spray coating, Wurster coating, Pan coating and co-extrusion, coacervation, spray drying and spray chilling; and optionally, wherein one or more beneficial liquid ingredients are co-granulated with one or more solid active ingredients in the form of solid granules, pellets, tablets, encapsulated granules, sachets, matrix beads and capsules.

One or more layers or coatings of an ionic strength responsive material is applied over on tablet cores. The coatings may be applied using standard coating methods analogous to those described to apply the barrier coating.

Beads, granules or multiparticulates may be prepared by analogous methods to those used to prepare tablets.

Barrier compositions prepared from one or more ASE and HASE polymers form impermeable barriers that surround, encapsulate and/or form a matrix with one or more active ingredients, providing sufficient structural support while inhibiting the release of the beneficial agent prior to the triggered dissolution or dispersion of the barriers of the device. Aqueous system refers to but not limited to a solution containing water as the principal liquid component (e.g., solutions of organic or inorganic substances particularly electrolytes and surfactant mixtures of substance in water). Typically the barrier composition totally surrounds, encapsulates and/or forms a matrix with the beneficial agent/active ingredient or forms an impermeable matrix of the barrier composition and the beneficial agent/active ingredient. The impermeable barrier membrane material has a combination of thickness and mechanical strength so that it will be sufficiently stable at predetermined system including but not limited to a heavy duty liquid (HDL) formulation or fabric laundry wash cycle and will rapidly disrupt and release the beneficial ingredients once the desired triggered release environment has been generated. Preferably the impermeable barrier membrane is 5 μm to 300 μm in thickness for household and personal care applications, such as fabric care laundry application. The impermeable barrier membrane may be a dense film, a composite membrane, asymmetric in structure, etc. The preferred particle size of the impermeable matrix beads of the barrier composition and the beneficial agent/active ingredient 20 to 5000 μm. Typically the device of the barrier composition material and the beneficial ingredients is composed of emulsion polymers and personal care and household care actives including but not limited to fabric care actives, fragrances and pharmaceutically beneficial agents.

In one preferred embodiment, the selected group of ASE and HASE polymers in any structural form may be used as the ionic strength trigger means; or in addition to an ionic strength trigger means, a pH, surfactant concentration level, temperature, mechanical force and the combinations of thereof, trigger means that maintains the integrity of the device until triggered by a solution of the desired conditions. The trigger device may be for example an impermeable dense coating membrane or an impermeable matrix. Preferably, the trigger device provides sufficient structural support and optionally, is impermeable to water, which inhibits the core from contacting with the aqueous system, and releasing the beneficial agent until triggered. Typically the trigger device is selected from a group of ASE, ASR and HASE barrier compositions surrounding the ingredients, the barrier substantially impermeable to releasing the active ingredients to the aqueous system and remaining insoluble in the aqueous system at a predetermined conditions, the barrier becoming soluble or dispersible or disintegrates in an aqueous system when the ionic strength changes; and in addition to ionic strength changes, changes in pH, temperature, surfactant concentration level, mechanical force and the combinations of thereof changed, effecting the rapid release of the active ingredients.

Typically the barrier materials are insoluble solids in an aqueous system. In a fabric care embodiment, the barrier materials are insoluble solids in an aqueous system including but not limited to fabric laundry wash cycle, and then they dissolve (or degrade, swell and disperse) when the ionic strength changes; and in addition to ionic strength changes, changes in pH, surfactant concentration level, temperature, mechanical forces and the combinations of thereof, in the system.

The devices of this invention having the above described desired characteristics may be made using the above described materials using the following processes and other conventional techniques and methods. Conventional techniques and typical pharmaceutical actives used for preparing pharmaceutical and/or personal care delivery devices include, for example, those disclosed in U.S. Pat. No. 5,358,502.

In one preferred embodiment of the present invention, one or more beneficial ingredients are encapsulated with impermeable membranes of one or more barrier compositions via conventional coating technology, including but not limited to fluid bed spray coating, Wurster coating, Pan coating, etc. The beneficial ingredients in liquid states can be co-granulated with other solid form active ingredients to form solid granules or tablets prior to coating process or it can be incorporated along or else together with other active ingredients into a capsule made from a water soluble polymer such as, for example, gelatin. A filled gelatin capsule of this kind of beneficial ingredients is then provided with the coating comprising of barrier compositions. The coating may be made sufficiently thick so that it will be sufficiently stable in wash cycle and rapidly dispersed to release beneficial ingredients in rinse cycle.

In order to ensure that the coating of the barrier compositions does not dissolve in the earlier steps of the washing or cleaning operation, for example, at the beginning of the main wash cycle in the case of machine laundry washing, the stability of the barrier compositions membrane can be controlled by adjusting the degree of neutralization of the barrier compositions so that it will be insoluble at the early beginning of the wash cycle when detergent has not dissolved, then upon neutralization by the aqueous system after the dissolution of detergent, the barrier membrane will remain stable in wash cycle and rapidly dissolved or dispersed in rinse cycle.

In another preferred embodiment of the present invention, one or more beneficial ingredients are encapsulated with impermeable membranes of one or more barrier compositions or an impermeable matrix of one or more beneficial ingredients and one or more barrier compositions via emulsion polymerization, suspension polymerization, and micro-suspension polymerization. Depending on which polymerization process is employed, the particle size of the final encapsulated particles or matrix particles is between 0.01 to 1000 μm.

In another preferred embodiment of the present invention, one or more beneficial ingredients are encapsulated with one or more barrier compositions to form polymeric matrix beads. The matrix beads have the same actives in the cores as are described above and surrounded by a solid polymer protective shell formed during the solidification process by either spray drying or spray chilling or by precipitating with inorganic salt solution such as CaCl₂ or Na₂SO₄. Likewise the beads are preferably about 10 to 5000 μm big. The matrix beads made of polymer barrier compositions and beneficial ingredients contain 5 to 80% polymer barrier composition, 5 to 75% beneficial ingredients and 0 to 10% aids including surfactants. Preferably, the matrix beads should contain 5 to 50% ASE barrier polymers, 20 to 75% beneficial ingredients and 0 to 10% aids including surfactants.

The device shape and dimensions can vary based on the particular application (e.g., tablets, beads or capsules). The shape and size may also vary depending on the application so that for example the tablet is suitable depending on the quantity and rate of beneficial agent releasing which vary based on the application. Preferably, the tablet is 0.5 to 20 mm in diameter and the beads are 5 μm to 5 mm in diameter. However, typical device dimensions range from about 1 cm to about 2.5 cm in length and about 0.3 cm to about 1 cm in diameter for personal care and household applications. For other applications, such as flavors, fragrances, and other active ingredients for household and personal care applications, shapes and sizes will be determined by the method of use and may be different from those listed above.

Triggered response compositions of the present invention have utility as regulated release devices for personal care, controlled release of active ingredients and pharmaceutical agents, sensors, imaging and diagnostic agents, separations, molecular recognition, tracing devices and molecular biological conjugate assays.

It should be understood that the invention is not limited to the particular embodiments shown and described herein, but that various changes and modifications may be made without departing from the spirit and scope hereof as defined by the following claims.

### EXAMPLE 1

Triggered Response of Thin Films of HASE Polymers:

Thin films cast on glass slides preparations: Polymer thin films with thickness of about 50 μm were prepared by first pre-neutralizing polymer emulsion to desired pH with 0.1 M NaOH aqueous solution, then casting the emulsions onto glass slides, and drying on a hot plate with the temperature range from 60 to 70° C. for 20 to 30 minutes.

Free standing films preparation: Polymer free standing films were prepared by casting 1 gram pre-neutralized emulsion onto an aluminum weighing pan and drying at 70° C. oven for 120 minutes. After the film was dry, free standing film with thickness of 100 to 200 μm was peel off from the aluminum substrate.

Beaker test: Thin films cast on glass slides were immersed into 0.6% Tide powder detergent solution and tap water with pH 8.5 (adjusted with NaOH), respectively. No mechanical agitation was applied in beaker test.

The response results of films with different compositions are summarized as following:

TABLE 1
PEL compositions suitable for laundry applications

| | Polymer | Stability in washing | | Solubility in rinse | |
|---|---|---|---|---|---|
| | compo- | conditions | | conditions | |
| | sition | Beaker | Terg | Beaker | Terg |
| Samples | pH | Test | Test | Test | Test |
| Composition A | 4.92 | stable | partially | partially | partially |
| 10 Sipomer | | | dissolved | dissolved | dissolved |
| BEM(ai)/60 | | | | | |
| MA/20 AA/ | | | | | |
| 10 MAA | | | | | |
| Composition B | 5.04 | stable | partially | dissolved | dissolved |
| 10 VSM-1/60 | | | dissolved | | |
| MA/20 AA/ | | | | | |
| 10 MAA | | | | | |
| Composition C | 5.2 | stable | stable | dissolved | dissolved |
| 10 VSM-1/60 | | | | | |
| EA/20 AA/ | | | | | |
| 10 MAA | | | | | |
| Composition D | 5.2 | very stable | stable | dissolved | dissolved |
| 10 VSM-1/60 | | | | | |
| EA/20 AA/10 | | | | | |
| MAA// | | | | | |
| 0.2 DAP | | | | | |
| Composition E | 5.5 | stable | stable | Did not | partially |
| 10 VSM-1/70 | | | | dissolve | dissolved |
| EA/20 AA | | | | | |

Sipomer BEM is supplied by Rhodia and its active ingredient is behenyl (EO)₂₅ methacrylate.

VSM-1 is a Rohm and Haas surfactant monomer, Cetyl-stearyl (EO)₂₀ methacrylate. MA is methyl acrylate, AA is acrylic acid, MAA is methacrylic acid, EA is ethyl acrylate, and DAP is diallyl phthalate. The term “dissolved” indicates no polymer particles larger than 100 mesh (@ 150 um) were collected after a washing cycle.

By changing the monomer selections, polymer charge density and degree of neutralization, the properties of polymer films can be tuned to be sufficiently stable in fabric laundry wash cycle and dissolve or dispersed in fabric laundry rinse cycle conditions.

### EXAMPLE 2

Free-Standing PEL Film Cubic Swelling Ratio Under Different Salt Concentrations

Experimental:

Free-standing films with thickness of 50 μm were cast from a composition (60BA/lOSty/12MMA/18MAA/0.5LOFA) at room temperature. The films (dimensions of 1×1 cm) were placed in NaCl aqueous solution at pH 12, the cubic swell ratio of each film was measured after it reaches equilibrium. The results were summarized in FIG. 1.

PEL films are stable in high ionic strength aqueous media and swell at lower ionic strength or upon dilution with water.

### EXAMPLE 3

Triggered Response of Free-Standing Films of PEL (Compositions D) with Different Degree of Neutralization.

Composition D emulsions were pre-neutralized with an aqueous solution of 0.2 M NaOH to different degree of neutralization, the triggered response of their correspondent free standing films were tested in Terg —O— Tometer at 40° C. for 20 minutes for wash cycle and at room temperature 5 minutes for rinse cycle under the following conditions:

Terg-O-Tometer test: Free standing films were tested in a Terg-O-Tometer. Test conditions are the following:

A: wash conditions:

Detergent concentration: 0.6% Tide powder detergent;

Temperature: 25° C.;

Agitation: 90 RPM;

Hardness of the wash water: 300 ppm.

Fabric added: 5 gram black cotton cloth.

0.2 gram of coagulated polymer films was dosed in the Terg pot and washed at 25° C. After wash, water was collected using a screen with pore size smaller than 200 mesh.

B: Rinse Conditions:

Temperature: Room temperature;

Agitation: 90 RPM;

Fabric added: 5 gram;

Time: 5 minutes.

Results are summarized in Table 2:

TABLE 2
Triggered Response of PEL Compositions D under
different degree of neutralizations

| Degree of | | Film | Stability | |
|---|---|---|---|---|
| neutralization | pH of | thickness | in | Solubility in |
| (%) | emulsion | (μm) | wash | rinse |
| 0 | 2.3 | 100 | Partially | Dissolved in 5 min. |
| | | | dissolved | |
| 2.5 | 3.8 | 50 | Partially | Dissolved in 5 min. |
| | | | dissolved | |
| 5 | 4.5 | 50 | Did not | Dissolved in 5 min. |
| | | | dissolve | |
| 7.5 | 4.8 | 50 | Did not | Dissolved in 5 min. |
| | | | dissolve | |
| 15 | 5.2 | 70 to 90 | Did not | Dissolved in 5 min. |
| | | | dissolve | |

BGDMA is butyleneglycol dimethacrylate.

The triggered response of the barrier membranes can be affected by both the degree of neutralization and the film forming property. When the degree of neutralization of the emulsion equal to or large than 5%, the correspondent emulsions possess better film forming property. Therefore, the resulting membranes exhibited better stability in the system tested above.

### EXAMPLE 4

Swell Rates of PEL (Composition D) as Thin Films Cast on Glass Slides Under Different Salt Concentrations and Ionic Strengths in Aqueous Solutions.

Experimental: samples were prepared under the conditions described in EXAMPLE 1. The swelling rate of the films was evaluated at ambient temperature and 40° C., and in 0.1 M and 0.001 M NaOH, NaCl and Na₂CO₃ aqueous solutions. FIGS. 2 and 3 summarize the results.

Temperature only had a minor effect on the swelling rate of the polymer films in NaCl and Na₂CO₃ solutions. At room temperature and 40° C., the swelling ratio of the films in these two solutions exhibited minimal changes. Temperature exhibited more stronger effect on the swelling rate of the film in 0.1M NaOH solution. At 40° C., it is impossible to accurately measure the weight of the polymer film after the film was swelled in NaOH solution for 15 minutes, because the film already partially dissolved in the solution. The film of composition D swelled five times fast in 0.1 M NaOH solution as compared to in NaCl and Na₂CO₃ solutions at the same concentration.

The swelling rates of PEL (composition D) films in 0.001M NaOH, NaCl and Na₂CO₃ aqueous solutions were different as compared to the swelling rates of the same films in 0.1 M NaOH, NaCl and Na₂CO₃ solutions. The films swelled rapidly in the initial five minutes in NaOH solution, then slowly dissolved as indicated by the weight loss noted in FIG. 3. The swelling rates of the films in NaCl and Na₂CO₃ solution increased noticeably in lower ionic strength environments initially and slowly dissolved afterwards.

The swell ratios decreased after the films were immerged in the solutions for 5 minutes, which indicate that the films either were partially dissolved or fell out of the slides.

### EXAMPLE 5

Controlled Release of Encapsulated Fragrance

Experimental: A PEL (Composition D) emulsion was mixed with fragrance formulations using a homogenizer, stable emulsion systems were obtained. Freestanding films were cast from the resulting polymer emulsion and fragrance formulation mixtures. The films were then placed in the following solutions to test the releasing of the fragrance.

a) in DI water;

b) in 1M NaCl solution

c) in 5M NaCl solution

The releasing rate of the fragrance from their polymeric matrix decreases significantly when the films were placed in salt solution. After one month, the freestanding film embedded with fragrance completely lost most of the fragrance when it was placed in DI water, the film itself was swelling and broke into pieces. The films placed in NaCl solution stay intact and still keep the fragrance.

### EXAMPLES 6-14

Preparation of Additional PEL Compositions

The polymer emulsions of interest are diluted to 20 weight percent polymers solids and completely neutralized by raising the pH of the aqueous emulsion to 10 with an aqueous solution of sodium hydroxide (2%). To the emulsions are added 100 ppm of FC-120 wetting aid and, if required, 10-20% of a coalescing agent on the polymer solids. The coalescing agent used typically is Dowanol® DE (diethylene glycol monomethyl ether). Some of the emulsion is cast on a glass plate and allowed to dry. The dried film is cut in to test strips. To run cubic swell ratios during the testing, the strips are cut 2 centimeters in length.

Film strips are tested for a triggered response to ionic strength and base strength (concentration) changes in 1.2% Bold® detergent solution and 0.6% Tide® detergent solution in vials in a water bath held at 60° C. for at least 30 minutes. If the film is still intact after that time, 95% of the detergent solution in the vial is removed and replaced with tap water in order to assess how the film responds in water of neutral pH and relatively low ionic strength. Cubic swell ratios are measured after testing and are equal to the cubic ratio of the film length exposed to ions and bases to the original film length as cast, [final length/original length]³.

### EXAMPLE 6

The composition is a polyelectrolyte of 52.5 weight percent methyl methacrylate (MMA), 29.5 weight percent butyl acrylate (BA), 18 weight percent methacrylic acid (MAA) and 1.5 weight percent 3-mercaptopropionic acid (3-MPA). The polyelectrolyte is stable in an aqueous solution of NaOH of 2.5 M or greater and is triggered to swell/dissolve/disperse by lowering the concentration of NaOH to 1.0 M or less.

### EXAMPLE 7

The composition is a polyelectrolyte of 33 weight percent styrene (Sty), 35 weight percent butyl acrylate (BA), 18 weight percent methyl methacrylate (MMA) and 25 weight percent methacrylic acid (MAA). The polyelectrolyte is stable in an aqueous solution of NaOH of 1.0 M or greater and is triggered to swell/dissolve/disperse by lowering the concentration of NaOH to 0.1 M or less.

### EXAMPLE 8

An aqueous solution of composition 60 BA/21MMA/102-ethyl hexyl acrylate (HEMA)/9MAA (1% backbone cross-linking with zinc ions), was adjusted to pH 10.5 using aqueous 2% NaOH solution. Film fell apart at 60° C. in 1.2% Bold in 4 min. and disintegrated in 8 min. Film was close to degrading at 60° C. in 0.6% Tide after 30 min. Fell apart upon 20:1 dilution (vol:vol) yet did not dissolve or disintegrate. Film fell apart at 60° C. in 0.6% Bold in 20 min. and disintegrated in 30 min.

### EXAMPLE 9

An aqueous solution of composition 60 BA/21MMA/10 HEMA/9MAA (1% backbone cross-linking with calcium ions), was adjusted to pH 11.0 using aqueous 2% NaOH solution. Film was delicate/fragile at 600 C in 1.2% Bold after 20 min. and disintegrated in 30 min. Film was delicate/fragile at 60° C. in 0.6% Tide after 35 min. Fell apart upon 20:1 dilution (vol:vol) yet did not dissolve or disintegrate.

### EXAMPLE 10

An aqueous solution of composition 60 BA/21MMA/10 HEMA/9MAA (1% backbone cross-linking with magnesium ions), was adjusted to pH 10.5 using aqueous 2% NaOH solution. Film disintegrated at 60° C. in 1.2% Bold after 30 min. Film was swollen but still remained intact at 60° C. in 0.6% Tide after 35 min. Fell apart upon 20:1 dilution (vol:vol).

### EXAMPLE 11

An aqueous solution of composition containing 65 weight percent of 60 BA/21MMA/10 HEMA/9MAA and 35 weight percent of 80 Sty/10MMA/10AA (1% backbone cross-linking with zinc ions), was adjusted to pH 10.5 using aqueous 2% NaOH solution. Film fell apart at 60° C. in 1.2% Bold after 20 min. and disintegrated in 35 min. Film was swollen but remained intact 60° C. in 0.6% Tide after 35 min. Mild agitation caused upon 20:1 dilution (vol:vol) caused the film to break into 20 pieces. No dissolution or disintegration.

### EXAMPLE 12

An aqueous solution of composition containing 65 weight percent of 60 BA/21MMA/10 HEMA/9MAA and 35 weight percent of 80 Sty/10MMA/10AA (1% backbone cross-linking with calcium ions), was adjusted to pH 11.0 using aqueous 2% NaOH solution. Film swelled upon 20:1 dilution (vol:vol) yet retained integrity. Cubic swell ratio (CSR) in 0.6% Tide wash, CSR=4.91. CSR in Tide rinse water=6.86. CSR in 1.2% Bold wash =3.38. CSR in Bold rinse water=5.36.

### EXAMPLE 13

An aqueous solution of composition containing 65 weight percent of 60 BA/21MMA/10 HEMA/9MAA and 35 weight percent of 80 Sty/10MMA/10AA (1% backbone cross-linking with magnesium ions), was adjusted to pH 10.5 using aqueous 2% NaOH solution. Film swelled upon 20:1 dilution (vol:vol) yet retained integrity. Cubic swell ratio (CSR) in 0.6% Tide wash, CSR=6.86. CSR in Tide rinse water=27.0. CSR in 1.2% Bold wash =4.33. CSR in Bold rinse water=9.94.

### EXAMPLE 14

An aqueous solution of composition containing 50 weight percent of 35 BA/33Sty/7MMA/25MAA and 50 weight percent of 60BA/21MMA/10HEMA/10AA (1% backbone cross-linking with zinc ions), was adjusted to pH 10.5 using aqueous 2% NaOH solution. An aqueous solution of composition JLE-1983 (1% backbone cross-linking with calcium ions), was adjusted to pH 11.0 using aqueous 2% NaOH solution. An aqueous solution of composition JLE-1980 (1% backbone cross-linking with magnesium ions), was adjusted to pH 10.5 using aqueous 2% NaOH solution. The zinc cross-linked film disintegrated at 60° C. in 1.2% Bold in 20 min. The magnesium cross-linked film disintegrated at 60° C. in 1.2% Bold after 35 min. The calcium cross-linked film retained integrity at 600 C in 1.2% Bold after 35 min. All films have good integrity and remained intact at 600 C in 0.6% Tide after 35 min. All four non-disintegrating films swelled much more in rinse waterupon 20:1 dilution (vol:vol) yet retained integrity and remained intact.

Cubic swell ratios are presented for selected ionic strength and base responsive polyelectrolytic compositions in Table 3.

TABLE 3
Cubic Swell Ratios for Ionic Strength and Base Responsive
Polyelectrolytic Compositions

| Polyelectrolyte | | |
|---|---|---|
| Wt. % | | |
| Monomers | Swelling Solution | CSR |
| 40 Sty/35 BA/ | 2.5 M NaOH | 1.46 |
| 9MMA/16MAA | 1.0 M NaOH | 1.64 |
| (Zn²⁺and NH₃ | 0.25 M NaOH | 2.89 |
| free) | 0.1 M NaOH | 3.91 |
| | Tap water | 11.0 |
| 40 Sty/35 BA/ | 2.5 M NaOH | 1.52 |
| 9MMA/16MAA | 1.0 M NaOH | 1.73 |
| (1% n-DDM) | 0.1 M NaOH | 8 (film disintegrated) |
| 40 Sty/35 BA/ | 1.0 M NaOH | 1.73 |
| 9MMA/16MAA | 0.1 M NaOH | Film dissolved |
| (1.5% n-DDM) | | |
| 20 Sty/35 BA/ | 2.5 M NaOH | 4.1 |
| 29MMA/16MAA | 0.1 M NaOH | Film dissolved |
| (1.5% n-DDM) | | |
| 20 Sty/35 BA/ | 2.5 M NaOH | 1.62 |
| 29MMA/16MAA | 1.0 M NaOH | 3.21 |
| | 0.1 M NaOH | 6.33 |
| | Tap water | > 30 |
| 40 Sty/35 BA/ | 2.5 M NaOH | 1.33 |
| 7MMA/18MAA | 1.0 M NaOH | 1.42 |
| | 0.1 M NaOH | 4.1 |
| | Tap water | 11.02 |
| 41 Sty/34 BA/ | 2.5 M NaOH | 1.33 |
| 9MMA/16MAA | 1.0 M NaOH | 1.62 |
| | 0.1 M NaOH | 3.55 |
| | Tap water | 9.6 |
| 33 Sty/35 BA/ | 2.5 M NaOH | 1.39 |
| 7MMA/16MAA | 1.0 M NaOH | 2.46 |
| (1% LOFA) | 0.1 M NaOH | 7.59 |
| | Tap water | > 100 |
| 32 Sty/35 BA/ | 2.5 M NaOH | 1.52 |
| 12MMA/21MAA | 1.0 M NaOH | 2.15 |
| (0.5% LOFA) | 0.1 M NaOH | 8.62 (dissolved) |
| | Tap water | dissolved |
| 33 Sty/35 BA/ | 2.5 M NaOH | 1.71 |
| 7MMA/25MAA | 1.0 M NaOH | 2.33 |
| (0.5% LOFA) | 0.1 M NaOH | Rapidly dissolved |
| JLE-1937 | 2.5 M NaOH | 1.16 |
| With 37 wt. % | 1.0 M NaOH | 1.62 |
| gelatin | 0.1 M NaOH, film pre- | 4.1 |
| | neutralized | |
| | 0.1 M NaOH, film un- | 4.1 |
| | neutralized | |
| | Tap water | 17.6 |

n-DDM is n-dodecylmercaptan, LOFA is linseed oil fatty acid.
Rhoplex ® B-1604 is a product of Rohm and Haas Company.

We claim:

1. A triggered response composition comprising: one or more polyelectrolytes in contact with a liquid medium that is stable and that exhibits one or more chemical/physical responses; wherein the chemical/physical response is triggered upon one or more ionic strength changes to the liquid medium; wherein the polyelectrolyte is one or more acidic homopolymers, copolymers, polymer blends and salts thereof one or more basic homopolymers, copolymers, polymer blends and salts thereof; and one or more amphoteric homopolymers, copolymers, polymer blends and salts thereof.

2. The triggered response composition according to claim 1 wherein the polyelectrolyte is one or more alkali soluble or swellable polymers comprising: (a) 15-70 weight percent of one or more acidic monomers; (b) 15-80 weight percent of one or more non-ionic vinyl monomers; (c) 0-30 weight percent of one or more non-ionic vinyl surfactant monomers; and, optionally, (d) 0-5 weight percent of one or more polyethylenically unsaturated monomers; and wherein the chemical/physical response of the polymers as a function of ionic strength changes is dependent on one or more parameters selected from the group consisting of (i) the type and amounts of acidic monomers, (ii) the degree of neutralization of the acidic monomers, (iii) the type and amounts of non-ionic vinyl surfactant monomers, (iv) the type and amounts of non-ionic vinyl monomers, (v) the type and amounts of polyethylenically unsaturated monomers, (vi) the pH of the aqueous system and (vii) combinations thereof; wherein the chemical/physical response of the composition is selected from selected from dispersing, disintegrating, dissolving, destabilizing, deforming, swelling, softening, flowing and combinations thereof; and wherein the liquid medium is an aqueous system.

3. A triggered response barrier composition comprising: one or more polyelectrolytes in contact with a liquid medium, wherein the barrier composition surrounds, encapsulates or forms a matrix with one or more active ingredients; wherein the barrier composition is stable in the liquid medium; wherein the barrier exhibits one or more chemical/physical responses that is triggered upon one or more ionic strength changes to the liquid medium; and wherein the barrier composition is capable of releasing the active ingredients to the liquid medium as a result of the triggered response; and wherein the polyelectrolyte is one or more acidic homopolymers, copolymers, polymer blends and salts thereof; one or more basic homopolymers, copolymers, polymer blends and salts thereof; and one or more amphoteric homopolymers, copolymers, polymer blends and salts thereof.

4. The triggered response barrier composition according to claim 3 wherein the barrier composition is in the form of a film, wherein the liquid medium is an aqueous system, and wherein the chemical/physical response of the composition is selected from selected from dispersing, disintegrating, dissolving, destabilizing, deforming, swelling, softening, flowing and combinations thereof.

5. The triggered response barrier composition according to claim 4, wherein the barrier composition is stable and insoluble in an aqueous system at relatively high ionic strength and wherein the composition disperses, dissolves, swells or disintegrates in an aqueous system at relatively low ionic strength, and wherein the chemical/physical response of the polymers is a function of changes in one or more parameters in addition to ionic strength selected from the group consisting of: ion concentration, surfactant concentration, acid strength and concentration, base strength and concentration, pH, buffer strength and capacity, temperature, hydrogen bonding, hydrogen bonding solvents, organic solvents, osmotic pressure, dilution, viscosity, electrochemical potential, conductivity, ion mobility, charge mobility, diffusion, surface area, mechanical forces, radiation and combinations thereof.

6. A device for the triggered release of one or more active ingredients to a liquid medium comprising:
(d) one or more active ingredients;
(e) one or more additives; and
(f) a barrier composition comprising one or more ionic strength responsive polyelectrolytes;
wherein the barrier composition surrounds, encapsulates or forms a matrix with one or more active ingredients; wherein the barrier composition is stable in the liquid medium; wherein the polyelectrolyte is one or more acidic homopolymers, copolymers, polymer blends and salts thereof; one or more basic homopolymers, copolymers, polymer blends and salts thereof; and one or more amphoteric homopolymers, copolymers, polymer blends and salts thereof; wherein the barrier exhibits one or more chemical/physical responses selected from dispersing, disintegrating, dissolving, destabilizing, swelling, softening, flowing and combinations thereof; wherein the chemical/physical response of the composition is triggered upon one or more ionic strength changes to the aqueous system; and wherein the device is capable of releasing the active ingredients to the aqueous system as a result of the triggered response of the barrier composition.

7. A process for triggering the release of one or more active ingredients to a liquid medium comprising the steps of:
(c) Surrounding, encapsulating or forming a matrix with one or more active ingredients with an ionic strength responsive barrier composition, the barrier being substantially impermeable to releasing the active ingredients to the liquid medium and remaining insoluble in the liquid medium; and
(d) altering the ionic strength of the liquid medium;
wherein the polyelectrolyte is one or more acidic homopolymers, copolymers, polymer blends and salts thereof; one or more basic homopolymers, copolymers, polymer blends and salts thereof; and one or more amphoteric homopolymers, copolymers, polymer blends and salts thereof; and wherein the barrier composition disperses, disintegrates, dissolves or swells and becomes substantially permeable, thereby triggering the release of the active ingredients into the liquid medium.

8. The process according to claim 7 wherein a device for the triggered release of one or more active ingredients to an aqueous system is prepared, the device comprising:
(a) one or more active ingredients;
(b) one or more additives; and
(c) a barrier composition comprising one or more ionic strength responsive polyelectrolytes;
wherein the barrier composition surrounds, encapsulates or forms a matrix with one or more active ingredients; wherein the barrier composition is stable and insoluble in an aqueous system at relatively high ionic strength; wherein the barrier exhibits one or more chemical/physical responses selected from dispersing, disintegrating, dissolving, destabilizing, swelling, softening, flowing and combinations thereof; wherein the chemical/physical response of the composition is triggered upon one or more ionic strength changes to the aqueous system; wherein the device is capable of releasing the active ingredients to the aqueous system as a result of the triggered response of the barrier composition; wherein the device is prepared using coating technology selected from the group consisting of fluid bed spray coating, Wurster coating, Pan coating and co-extrusion, coacervation, spray drying and spray chilling; and optionally, wherein one or more beneficial liquid ingredients are co-granulated with one or more solid active ingredients in the form of solid granules, pellets, tablets, encapsulated granules, sachets, matrix beads and capsules. in an altered or separate aqueous system
